SEQUENCE LISTING (80 sequences)

SEQ ID NO: 1 - protein, 5 aa, Homo sapiens
RRASA

SEQ ID NO: 2 - protein, 4 aa, Homo sapiens
NLTK

SEQ ID NO: 3 - protein, 5 aa, Homo sapiens
QNLTK

SEQ ID NO: 4 - protein, 6 aa, Homo sapiens
FQNLTK

SEQ ID NO: 5 - protein, 5 aa, Homo sapiens
KLLQE

SEQ ID NO: 6 - protein, 6 aa, Homo sapiens
KLLQEE

SEQ ID NO: 7 - protein, 7 aa, Homo sapiens
KLLQEEN

SEQ ID NO: 8 - protein, 8 aa, Homo sapiens
KLLQEENC

SEQ ID NO: 9 - protein, 9 aa, Homo sapiens
KLLQEENCD

SEQ ID NO: 10 - protein, 10 aa, Homo sapiens
KLLQEENCDI

SEQ ID NO: 11 - protein, 11 aa, Homo sapiens
KLLQEENCDIF

SEQ ID NO: 12 - protein, 12 aa, Homo sapiens
KLLQEENCDIFQ

SEQ ID NO: 13 - protein, 13 aa, Homo sapiens
KLLQEENCDIFQN

SEQ ID NO: 14 - protein, 14 aa, Homo sapiens
KLLQEENCDIFQNL

SEQ ID NO: 15 - protein, 15 aa, Homo sapiens
KLLQEENCDIFQNLT

SEQ ID NO: 16 - protein, 16 aa, Homo sapiens
KLLQEENCDIFQNLTK

SEQ ID NO: 17 - protein, 6 aa, Homo sapiens
FKLLQE

SEQ ID NO: 18 - protein, 7 aa, Homo sapiens
GFKLLQE

SEQ ID NO: 19 - protein, 8 aa, Homo sapiens
VGFKLLQE

SEQ ID NO: 20 - protein, 9 aa, Homo sapiens
AVGFKLLQE

SEQ ID NO: 21 - protein, 10 aa, Homo sapiens
LAVGFKLLQE

SEQ ID NO: 22 - protein, 11 aa, Homo sapiens
HLAVGFKLLQE

SEQ ID NO: 23 - protein, 12 aa, Homo sapiens
HHLAVGFKLLQE

SEQ ID NO: 24 - protein, 13 aa, Homo sapiens
NHHLAVGFKLLQE

SEQ ID NO: 25 - protein, 14 aa, Homo sapiens
ENHHLAVGFKLLQE

SEQ ID NO: 26 - protein, 15 aa, Homo sapiens
HLAVGFKLLQEENCD

SEQ ID NO: 27 - protein, 17 aa, Homo sapiens
FKLLQEENCDIFQNLTK

SEQ ID NO: 28 - protein, 18 aa, Homo sapiens
GFKLLQEENCDIFQNLTK

SEQ ID NO: 29 - protein, 19 aa, Homo sapiens
VGFKLLQEENCDIFQNLTK

SEQ ID NO: 30 - protein, 20 aa, Homo sapiens
AVGFKLLQEENCDIFQNLTK

SEQ ID NO: 31 - protein, 21 aa, Homo sapiens
LAVGFKLLQEENCDIFQNLTK

SEQ ID NO: 32 - protein, 22 aa, Homo sapiens
HLAVGFKLLQEENCDIFQNLTK

SEQ ID NO: 33 - protein, 23 aa, Homo sapiens
HHLAVGFKLLQEENCDIFQNLTK

SEQ ID NO: 34 - protein, 24 aa, Homo sapiens
NHHLAVGFKLLQEENCDIFQNLTK

SEQ ID NO: 35 - protein, 25 aa, Homo sapiens
ENHHLAVGFKLLQEENCDIFQNLTK

SEQ ID NO: 36 - protein, 25 aa, Artificial sequence
  Synthetic sequence Peptide 906
ENHHLAVGFKLLQEENSDIFQNLTK

SEQ ID NO: 37 - protein, 25 aa, Artificial sequence
  Synthetic sequence Peptide control
KELGAINHEFDLSHNFQTQKLNLVE

SEQ ID NO: 38 - protein, 12 aa, Homo sapiens
ENHHLAVGFKLL

SEQ ID NO: 39 - protein, 11 aa, Homo sapiens
ENHHLAVGFKL

SEQ ID NO: 40 - protein, 10 aa, Homo sapiens
ENHHLAVGFK

SEQ ID NO: 41 - protein, 5 aa, Homo sapiens
VGFKL

SEQ ID NO: 42 - protein, 5 aa, Homo sapiens
GFKLL

SEQ ID NO: 43 - protein, 5 aa, Homo sapiens
FKLLQ

SEQ ID NO: 44 - protein, 4 aa, Homo sapiens
GFKL

SEQ ID NO: 45 - protein, 4 aa, Homo sapiens
FKLL

SEQ ID NO: 46 - protein, 7 aa, Homo sapiens
IFQNLTK

SEQ ID NO: 47 - protein, 5 aa, Homo sapiens
FQNLT

SEQ ID NO: 48 - protein, 4 aa, Homo sapiens
FQNL

SEQ ID NO: 49 - protein, 6 aa, Artificial sequence
  Synthetic peptide
FQNLTA

SEQ ID NO: 50 - protein, 6 aa, Artificial sequence
  Synthetic peptide
FQNLTN

SEQ ID NO: 51 - protein, 6 aa, Artificial sequence
  Synthetic peptide
FQNLTD

SEQ ID NO: 52 - protein, 6 aa, Artificial sequence
  Synthetic peptide
FQNLTQ

SEQ ID NO: 53 - protein, 6 aa, Artificial sequence
  Synthetic peptide
FQNLTE

SEQ ID NO: 54 - protein, 6 aa, Artificial sequence
  Synthetic peptide
FQNLTG

SEQ ID NO: 55 - protein, 6 aa, Artificial sequence
  Synthetic peptide
FQNLTC

SEQ ID NO: 56 - protein, 6 aa, Artificial sequence
  Synthetic peptide
FQNLTM

SEQ ID NO: 57 - protein, 6 aa, Artificial sequence
  Synthetic peptide
FQNLTF

SEQ ID NO: 58 - protein, 6 aa, Artificial sequence
  Synthetic peptide
FQNLTP

SEQ ID NO: 59 - protein, 6 aa, Artificial sequence
  Synthetic peptide
FQNLTW

SEQ ID NO: 60 - protein, 6 aa, Artificial sequence
  Synthetic peptide
FQNLTY

SEQ ID NO: 61 - protein, 6 aa, Artificial sequence
  Synthetic peptide
FQNLTS

SEQ ID NO: 62 - protein, 6 aa, Artificial sequence
  Synthetic peptide
FQNLTL

SEQ ID NO: 63 - protein, 6 aa, Artificial sequence
  Synthetic peptide
FQNLTH

SEQ ID NO: 64 - protein, 6 aa, Artificial sequence
  Synthetic peptide
FQNLTT

SEQ ID NO: 65 - protein, 6 aa, Artificial sequence
  Synthetic peptide
FQNLTV

SEQ ID NO: 66 - protein, 6 aa, Artificial sequence
  Synthetic peptide
FQNLTI

SEQ ID NO: 67 - protein, 6 aa, Artificial sequence
  Synthetic peptide
FQNLTR

SEQ ID NO: 68 - protein, 745 aa, Homo sapiens
MAQQTSPDTLTVPEVDNPHCPNPWLNEDLVKSLRENLLQHEKSKTARKSVSPKLSPVISPRNSPRLLRRMLLSSNIPKQRRFTVAHTCFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 69 - protein, 160 aa, Homo sapiens
MEIPVPVQPSWLRRASAPLPGLSAPGRLFDQRFGEGLLEAELAALCPTTLAPYYLRAPSVALPVAQVPTDPGHFSVLLDVKHFSPEEIAVKVVGEHVEVHARHEERPDEHGFVAREFHRRYRLPPGVDPAAVTSALSPEGVLSIQAAPASAQAPPPAAAK

SEQ ID NO: 70 - DNA, 32 bp, Artificial sequence
  Synthetic sequence Primer
gcacaagcttatggagatccctgtgcctgtgc

SEQ ID NO: 71 - DNA, 31 bp, Artificial sequence
  Synthetic sequence Primer
gcacaagcttcttggctgcggctggcggtgg

SEQ ID NO: 72 - protein, 6 aa, Artificial sequence
  Synthetic peptide
YQNLTA

SEQ ID NO: 73 - protein, 5 aa, Homo sapiens
YQNLT

SEQ ID NO: 74 - protein, 6 aa, Artificial sequence
  Synthetic peptide
YQNLTR

SEQ ID NO: 75 - protein, 6 aa, Artificial sequence
  Synthetic peptide
YQNLTK

SEQ ID NO: 76 - protein, 5 aa, Artificial sequence
  Synthetic peptide
GFRLL

SEQ ID NO: 77 - protein, 5 aa, Artificial sequence
  Synthetic peptide
KFKLL

SEQ ID NO: 78 - protein, 5 aa, Artificial sequence
  Synthetic peptide
RFKLL

SEQ ID NO: 79 - protein, 5 aa, Artificial sequence
  Synthetic peptide
RFRLL

SEQ ID NO: 80 - protein, 5 aa, Artificial sequence
  Synthetic peptide
GFALL

(19) United States
(12) Patent Application Publication
(10) Pub. No.: US 2013/0209439 A1
(43) Pub. Date: Aug. 15, 2013

(54) INHIBITORS OF PDE4 BINDING TO HSP20

(71) Applicants: George S. Baillie, Glasgow (GB); Miles Houslay, Glasgow (GB); Jon Day, Glasgow (GB)

(72) Inventors: George S. Baillie, Glasgow (GB); Miles Houslay, Glasgow (GB); Jon Day, Glasgow (GB)

(73) Assignee: THE UNIVERSITY COURT OF THE UNIVERSITY OF GLASGOW, Glasgow (GB)

(21) Appl. No.: 13/878,404

(22) PCT Filed: Oct. 7, 2011
(86) PCT No.: PCT/GB11/01457

§ 371 Date: Apr. 8, 2013

(30) Foreign Application Priority Data
Oct. 8, 2010 (GB) 1017056.1

(51) Int. Cl. C12N 9/16; G01N 33/68

(52) U.S. Cl. CPC C12N 9/16; G01N 33/68
USPC 424/94.6; 435/7.1; 435/7.92; 514/1.1; 514/21.91; 514/21.9; 514/21.8; 514/21.3; 514/21.4; 435/375; 514/17.8; 514/17.7; 514/15.1; 514/13.5; 514/16.4; 514/18.9; 506/9; 536/23.1; 435/320.1; 435/369; 562/564; 530/331; 530/330; 530/329

(57) ABSTRACT

The present invention provides methods and materials for use in increasing HSP20 activation in a biological system, for example by increasing phosphorylation of Ser16 of HSP20. In one aspect, the invention provides a method for increasing HSP20 activation in a biological system, comprising contacting the system with an antagonist capable of inhibiting PDE4 binding to HSP20, the antagonist comprising or consisting essentially of a fragment of PDE4 or an analogue thereof. In a further aspect the invention provides a method of screening for an agent able to increase activation of HSP20. A preferred antagonist has a C-terminal lysine residue.

### CROSS-REFERENCE TO RELATED APPLICATIONS

This patent application claims the benefit of priority of International Application No. PCT/GB2011/001457, filed Oct. 7, 2011, which claims the benefit of priority of Great Britain Patent Application No. 1017056.1, filed Oct. 8, 2010 both of which are incorporated herein by reference in their entireties.

### FIELD OF THE INVENTION

The invention relates to the newly discovered interaction between PDE4 and HSP20, and in particular to antagonists that inhibit this interaction, methods of using these antagonists to increase HSP20 activation, treatment of pathological conditions with these antagonists, and methods of identifying new antagonists.

### BACKGROUND TO THE INVENTION

The small heat shock proteins (sHSPs) are a highly conserved family of molecular chaperones that are ubiquitously expressed throughout nature¹. The expression of sHSPs² depends on factors such as changes in physical/chemical stress, development and pathological status³. Increased expression of sHSPs results in cellular protection and often allows cells to better tolerate changes to their local environment triggered by physiological stresses during disease. Of the 10 known sHSPs⁴, HSP20 (also known as HspB6) has recently been described as a protective agent against a number of diseases of the brain including cerebral amyloid angiopathy⁵, Alzheimer's disease⁶ and forebrain ischemia⁷. A functional role for HSP20 is also well established in the context of cardiac disease, where it is regarded as acting as an innate protector during cardiac ischemia/reperfusion⁸, chronic β-adrenergic stimulation^{9, 10}, pharmacological treatment by doxorubicin¹¹, vasospasm and platelet aggregation¹², endotoxin induced myocardial dysfunction¹³, and congestive heart failure¹⁴. Functionally, HSP20 confers cardiac protection by a number of diverse mechanisms including the suppression of NF-k-B signalling¹³, inhibition of caspase activity¹³, promotion of autophagy² stabilisation of the cytoskeleton¹⁵, reduction of myocardial necrosis and apoptosis⁸, increase in myocyte shortening rate via increases in calcium uptake¹⁶ and enhancement of Akt/PKB signalling¹¹. The protective potential of HSP20 is only realised after it has been phosphorylated on a PKA/PKG consensus motif within the N-terminal of the protein following a stimulus that increases cyclic nucleotide concentration within cells¹⁷. Diminished phosphorylation at Ser16 abrogates the protective effect of HSP20¹⁸ and overexpression of a constitutively non-phosphorylated mutant (S16A), increases susceptibility to ex vivo ischemia/reperfusion injury², induces cell necrosis² and increases infarct areas². Conversely, a constitutively phosphorylated mutant (S16D) confers full protection from apoptosis via inhibition of caspase 3¹⁰.

Although both PKA and PKG can, potentially, effect the cardio-protective phosphorylation of HSP20 at Ser16, this residue occurs within a classical and robust consensus site (13-RRASA-17) (SEQ ID NO: 1) for PKA phosphorylation, which is of the form RRxS-hydrophobic amino acid. Indeed, there is comprehensive evidence to suggest that, in response to β-agonists, PKA is the sole kinase responsible for phosphorylation at this site. Thus, pre-incubation with either peptide¹⁹ or pharmacological inhibitors of PKA^{20, 21} clearly ablates Ser16 phosphorylation of HSP20. Cell-permeable, phospho-peptide analogues that encompass the PKA phospho-site of HSP20 (Ser16)²² or the entire phosphorylated protein sequence²³, can mimic some of the protective functions of chaperone. However, the functional use of these peptides is limited by the difficulties associated with their entry into cells. Treatment with the adenylate cyclase activator, forskolin, has provided a commonly employed means to induce the phosphorylation of HSP20 by PKA in order to allow evaluation of the mechanics behind this modification¹⁹. However, these studies have not provided detailed functional information due to the nature of the non-specific, global increases in cAMP triggered by the concomitant activation of the complete adenylate cyclase pool.

Recent studies using genetically encoded cAMP reporter constructs in cardiac myocytes have suggested that certain cAMP phosphodiesterase (PDE) inhibitors can raise localised cAMP concentrations in defined cellular locations, in response to different agonists²⁴. Specifically, these studies suggest a robust functional coupling of one family of PDEs, namely PDE4, with the catecholamine induced cAMP response associated with β-adrenergic signalling²⁵. PDE4 is of much interest as selective inhibitors provide potential therapeutics for various diseases²⁶. Over 20 different isoforms of PDE4 exist via the mRNA splicing of 4 PDE4 genes (A, B, C, D) and each is characterised by a unique N-terminal region that contains a targeting sequence of which one critical function is to direct each isoform to specific intracellular locations/signalling complexes²⁷. Such targeting has functional relevance in the heart where PDE4 isoforms form complexes to underpin signal specific responses.

### SUMMARY OF THE INVENTION

The inventors have found that PDE4 isoforms can bind to HSP20, and have identified a previously unknown HSP20-binding region of PDE4. An antagonist comprising this HSP20-binding region is able to antagonise the interaction between HSP20 and PDE4, resulting in increased levels of the phosphorylated or ‘activated’ form of HSP20, which gives rise to the protective effects of HSP20 described herein.

Without wishing to be bound by any particular theory, it is believed that antagonising the interaction between PDE4 and HSP20 inhibits a PDE4-dependent decrease in local cAMP concentration, thereby promoting phosphorylation of HSP20 on Ser-16 by PKA.

This finding has clear implications for all pathological conditions in which activated HSP20 has a beneficial effect.

As discussed above, activation of HSP20 can equate to phosphorylation at Ser16 of HSP20.

In a first aspect, the present invention provides a method for increasing HSP20 activation in a biological system, comprising contacting the system with an antagonist capable of inhibiting PDE4 binding to HSP20.

The antagonist may comprise an HSP20-binding moiety capable of inhibiting PDE4 binding to HSP20. The HSP20-binding moiety may be a protein moiety. The term “protein” is not to be taken to imply any particular maximum or minimum size for the HSP20-binding moiety. Instead, it simply signifies that the HSP20-binding moiety is proteinaceous in nature, i.e. composed of amino acids linked by peptide bonds. It will be understood that this is intended to encompass protein derivatives such as glycoprotein and lipoprotein moieties.

As discussed in more detail below, a suitable HSP20-binding moiety may comprise a fragment of a PDE4 or an analogue thereof which is capable of binding to HSP20. Such molecules may be capable of binding to the same site on HSP20 as full-length catalytically active PDE4 and thus competitively inhibiting (e.g. preventing or disrupting) binding of active PDE4 to HSP20.

Alternatively, the binding moiety may comprise an antibody binding site specific for HSP20 which is capable of inhibiting binding of PDE4 to HSP20. Thus it may bind to the same site as PDE4, or to a different site, and may (for example) inhibit binding of PDE4 competitively, by steric interference, or by inducing a conformational change in HSP20 which inhibits PDE4 binding.

It will be apparent to the skilled person that other types of antagonists may also be suitable. Examples include nucleic acids, e.g. an aptamer specific for HSP20, which may be capable of inhibiting PDE4 binding in a similar manner to an antibody binding domain. Other examples include carbohydrates and small molecules.

When the antagonist is a fragment of PDE4 or an analogue thereof it may comprise a sequence represented by a formula selected from:

### X491-X492, X490-X491-X492, X489-X490-X491-X492, X488-X489-X490-X491-X492 or X487-X488-X489-X490-X491-X492,

or a fragment thereof capable of binding to HSP20, wherein
X487 is F or is substituted with any amino acid other than Y, optionally an uncharged amino acid or a non-polar amino acid;
X488 is Q or is substituted with any amino acid, optionally an uncharged amino acid or an uncharged polar amino acid;
X489 is N or is substituted with any amino acid, optionally an uncharged amino acid or an uncharged polar amino acid;
X490 is L or is substituted with any amino acid, optionally an uncharged amino acid or a non-polar amino acid;
X491 is T or is substituted with any amino acid, optionally an uncharged amino acid or an uncharged polar amino acid;

### X492 is K.

In a further embodiment, when the antagonist is a fragment of PDE4 or an analogue thereof it may comprise a sequence represented by the formula:

### X468-X469-X470-X471-X472-X473-X474-X475-X476-X477-X478-X479-X480-X481-X482-X483-X484-X485-X486-X486-X487-X488-X489-X490-X491-X492

or a fragment thereof capable of binding to HSP20, wherein
X468 is E or is substituted with any amino acid, e.g. an uncharged amino acid or a negatively charged amino acid;
X469 is N or is substituted with any amino acid, e.g. an uncharged amino acid, e.g. an uncharged polar amino acid;
X470 is H or is substituted with a positively charged amino acid;
X471 is H or is substituted with a positively charged amino acid;
X472 is L or is substituted with any amino acid, e.g. an uncharged amino acid, e.g. a non-polar amino acid;
X473 is A or is substituted with any amino acid, e.g. an uncharged amino acid, e.g. a non-polar amino acid;
X474 is V or is substituted with any amino acid, e.g. an uncharged amino acid, e.g. a non-polar amino acid;
X475 is G or is substituted with any amino acid, e.g. an uncharged amino acid, e.g. a non-polar amino acid;
X476 is F or is substituted with any amino acid, e.g. an uncharged amino acid, e.g. a non-polar amino acid;
X477 is K or is substituted with a positively charged amino acid;
X478 is L or is substituted with any amino acid, e.g. an uncharged amino acid, e.g. a non-polar amino acid;
X479 is L or is substituted with any amino acid, e.g. an uncharged amino acid, e.g. a non-polar amino acid;
X480 is Q or is substituted with any amino acid, e.g. an uncharged amino acid, e.g. an uncharged polar amino acid;
X481 is E or is substituted with a negatively charged amino acid or an uncharged amino acid;
X482 is E or is substituted with a negatively charged amino acid or an uncharged amino acid;
X483 is N or is substituted with any amino acid, e.g. an uncharged amino acid, e.g. an uncharged polar amino acid;
X484 is C or is substituted with any amino acid, e.g. an uncharged amino acid, e.g. a non-polar amino acid;
X485 is D or is substituted with a negatively charged amino acid;
X486 is I or is substituted with any amino acid, e.g. an uncharged amino acid, e.g. a non-polar amino acid;
X487 is F or is substituted with any amino acid, e.g. an uncharged amino acid, e.g. a non-polar amino acid;
X488 is Q or is substituted with any amino acid, e.g. an uncharged amino acid, e.g. an uncharged polar amino acid;
X489 is N or is substituted with any amino acid, e.g. an uncharged amino acid, e.g. an uncharged polar amino acid;
X490 is L or is substituted with any amino acid, e.g. an uncharged amino acid, e.g. a non-polar amino acid;
X491 is T or is substituted with any amino acid, e.g. an uncharged amino acid, e.g. an uncharged polar amino acid;
X492 is K or is substituted with any amino acid, e.g. a positively charged amino acid.

For the purposes of the above formulae, amino acids can be classified within the following groups:

I. Asp and Glu, and non-naturally occurring analogues thereof (negatively charged (or acidic) amino acids);
II. Arg, Lys and His, and non-naturally occurring analogues thereof (positively charged (or basic) amino acids);
III. Asn, Gln, Ser, Thr, Cys and Tyr, and non-naturally occurring analogues thereof (uncharged polar amino acids);
IV. Ala, Gly, Val, Leu, Ile, Pro, Phe, Met and Trp, and non-naturally occurring analogues thereof (non-polar amino acids).

A fragment capable of binding to HSP20 typically comprises at least 2, 3, 4 or 5 amino acids.

For example, it may comprise at least the sequence X491-X492. For example, it may comprise the sequence X490-X491-X492, X489-X490-X491-X492, X488-X489-X490-X491-X492 or X487-X488-X489-X490-X491-X492. It may therefore comprise the sequence TK, LTK, NLTK (SEQ ID NO: 2), QNLTK (SEQ ID NO: 3) or FQNLTK (SEQ ID NO: 4).

For example, it may comprise at least the sequence X477-X481. For example, it may comprise the sequence X477-X482, X477-X483, X477-X484, X477-X485, X477-X486. X477-X487, X477-X488. X477-X489, X477-X490, X477-X491 or X477-X492. It may therefore comprise the sequence KLLQE (SEQ ID NO: 5), KLLQEE (SEQ ID NO: 6), KLLQEEN (SEQ ID NO: 7), KLLQEENC (SEQ ID NO: 8), KLLQEENCD (SEQ ID NO: 9), KLLQEENCDI (SEQ ID NO: 10), KLLQEENCDIF (SEQ ID NO: 11), KLLQEENCDIFQ (SEQ ID NO: 12), KLLQEENCDIFQN (SEQ ID NO: 13), KLLQEENCDIFQNL (SEQ ID NO: 14), KLLQEENCDIFQNLT (SEQ ID NO: 15), KLLQEENCDIFQNLTK (SEQ ID NO: 16). For example, a fragment capable of binding to HSP20 may also comprise the sequence X476-X481, X475-X481, X474-X481, X473-X481, X472-X481, X471-X481, X470-X481, X469-X481, X468-X481. It may therefore comprise the sequence FKLLQE, (SEQ ID NO: 17) GFKLLQE (SEQ ID NO: 18), VGFKLLQE (SEQ ID NO: 19), AVGFKLLQE (SEQ ID NO: 20), LAVGFKLLQE (SEQ ID NO: 21), HLAVGFKLLQE (SEQ ID NO: 22), HHLAVGFKLLQE (SEQ ID NO: 23), NHHLAVGFKLLQE (SEQ ID NO: 24), ENHHLAVGFKLLQE (SEQ ID NO: 25). A fragment capable of binding to HSP20 may also comprise the sequence X471-X485, for example HLAVGFKLLQEENCD (SEQ ID NO: 26).

The numbering used in the above formula is intended simply to identify the corresponding residue in the full-length sequence of PDE4D5, and should not be taken to imply any particular length for the HSP20 binding moiety itself.

However it will be apparent that the HSP20 binding moiety may be present as part of a larger protein molecule, and may thus comprise further protein sequence N-terminal and/or C-terminal of the minimum sequence specified above. For example, it may comprise further PDE4 sequence, which may be from PDE4D5 or from another isoform of PDE4. In a preferred embodiment, the HSP20 binding moiety as described herein is at the C-terminus of a larger protein molecule. Optionally, the HSP20 binding moiety may be at the N-terminus of a larger protein molecule.

It will be understood by the skilled person that where the terms “comprising”, “comprises”, “including”, “includes” or the like are used herein with respect to the properties of a particular embodiment (for example, an antagonist, HSP20-binding moiety or fragment etc. comprising a particular amino sequence) the disclosure should be understood as applying mutatis mutandis to embodiments which “consist” or “consist essentially” of those properties (e.g. that amino acid sequence).

The HSP20 binding moiety may comprise 2, 3, 4 or 5 or more consecutive amino acids of PDE4, e.g. 6, 7, 8, 9, 10, 15, 20, 25, 30, 35, 40, 45, 50 or more consecutive amino acids of a PDE4, e.g. of PDE4D5.

Alternatively the HSP20 binding moiety may comprise a sequence of at least 2, 3, 4 or 5 amino acids, e.g. a sequence of 6, 7, 8, 9, 10, 15, 20, 25, 30, 35, 40, 45, 50 or more amino acids, which has at least 80% sequence identity with the corresponding sequence of PDE4, e.g. PDE4D5.

In some embodiments, the HSP20 binding moiety may have a maximum of 2, 3, 4, 5, 6, 7, 8, 9, 10, 15, 20, 25, 30, 35, 40, 45, 50 amino acids.

The corresponding sequence of PDE4 may comprise the sequence X477-X492. For example, the corresponding sequence of PDE4 may comprise the sequence X476-X492, X475-X492, X474-X492, X473-X492, X472-X492, X471-X492, X470-X492, X469-X492, X468-X492. It may therefore comprise the sequence KLLQEENCDIFQNLTK (SEQ ID NO: 16), FKLLQEENCDIFQNLTK (SEQ ID NO: 27), GFKLLQEENCDIFQNLTK (SEQ ID NO: 28), VGFKLLQEENCDIFQNLTK (SEQ ID NO: 29). AVGFKLLQEENCDIFQNLTK (SEQ ID NO: 30), LAVGFKLLQEENCDIFQNLTK (SEQ ID NO: 31). HLAVGFKLLQEENCDIFQNLTK (SEQ ID NO: 32), HHLAVGFKLLQEENCDIFQNLTK (SEQ ID NO: 33), NHHLAVGFKLLQEENCDIFQNLTK (SEQ ID NO: 34), ENHHLAVGFKLLQEENCDIFQNLTK (SEQ ID NO: 35). Thus the HSP binding moiety may have at least 80% sequence identity, e.g. at least 85%, 90% or 95% sequence identity with any one of these sequences.

The HSP20-binding moiety may comprise the amino acid sequence X477-X492, for example KLLQEENCDIFQNLTK (SEQ ID NO: 16).

In some embodiments

X487 is F or is substituted with an uncharged amino acid other than Y, or a non-naturally occurring analogue of phenylalanine, optionally a non-polar amino acid;
X488 is Q or is substituted with an uncharged amino acid, optionally an uncharged polar amino acid;
X489 is N or is substituted with an uncharged amino acid, optionally an uncharged polar amino acid;
X490 is L or is substituted with an uncharged amino acid, optionally a non-polar amino acid;
X491 is T or is substituted with an uncharged amino acid, optionally an uncharged polar amino acid;

### X492 is K.

In some embodiments

### X487 is F, M, I or L;

### X488 is Q or N;

### X489 is N or Q;

### X490 is L, I or M;

### X491 is T or S;

### X492 is K.

In some embodiments

X468 is E or is substituted with an uncharged amino acid or a negatively charged amino acid, e.g. a non-naturally occurring analogue of glutamate;
X469 is N or is substituted with an uncharged amino acid, e.g. an uncharged polar amino acid;
X470 is H or is substituted with a positively charged amino acid;
X471 is H or is substituted with a positively charged amino acid;
X472 is L or is substituted with an uncharged amino acid, e.g. a non-polar amino acid;
X473 is A or is substituted with an uncharged amino acid, e.g. a non-polar amino acid;
X474 is V or is substituted with an uncharged amino acid, e.g. a non-polar amino acid;
X475 is G or is substituted with an uncharged amino acid, e.g. a non-polar amino acid, e.g. a non-naturally occurring analogue of glycine;
X476 is F or is substituted with an uncharged amino acid or a non-naturally occurring analogue of phenylalanine, e.g. a non-polar amino acid;
X477 is K or is substituted with a positively charged amino acid;
X478 is L or is substituted with an uncharged amino acid, e.g. a non-polar amino acid;
X479 is L or is substituted with an uncharged amino acid, e.g. a non-polar amino acid;
X480 is Q or is substituted with an uncharged amino acid, e.g. an uncharged polar amino acid;
X481 is E or is substituted with a negatively charged amino acid or a non-naturally occurring analogue of glutamate;
X482 is E or is substituted with a negatively charged amino acid or a non-naturally occurring analogue of glutamate;
X483 is N or is substituted with an uncharged amino acid, e.g. an uncharged polar amino acid;
X484 is C or is substituted with an uncharged amino acid, e.g. a non-polar amino acid;
X485 is D or is substituted with a negatively charged amino acid;
X486 is I or is substituted with an uncharged amino acid, e.g. a non-polar amino acid;
X487 is F or is substituted with an uncharged amino acid or a non-naturally occurring analogue of phenylalanine, e.g. a non-polar amino acid;
X488 is Q or is substituted with an uncharged amino acid, e.g. an uncharged polar amino acid;
X489 is N or is substituted with an uncharged amino acid, e.g. an uncharged polar amino acid;
X490 is L or is substituted with an uncharged amino acid, e.g. a non-polar amino acid;
X491 is T or is substituted with an uncharged amino acid, e.g. an uncharged polar amino acid;
X492 is K or is substituted with a positively charged amino acid.

In some embodiments

### X468 is E or D;

### X469 is N or Q;

### X470 is H, K or R;

### X471 is H, K or R;

### X472 is L, I or M;

### X473 is A, G or S;

### X474 is V, I or A;

### X475 is G or A;

### X476 is F, M, I or L:

### X477 is K or R;

### X478 is L, I or M;

### X479 is L, I or M;

### X480 is Q or N;

### X481 is E or D;

### X482 is E or D;

### X483 is N or Q;

### X484 is C, A or G;

### X485 is D or E;

### X486 is I, V or L;

### X487 is F, M, I or L;

### X488 is Q or N;

### X489 is N or Q;

### X490 is L, I or M;

### X491 is T or S;

### X492 is K or R.

It may be desirable that the sequence X468-X492 or fragment thereof contains a certain minimum number of residues which are identical to those in the corresponding PDE4 sequence. For example, at least 1, 2, 3, 4, 5, 6, 7, 8, 9, 10, 11, 12, 13, 14, 15, 16, 17, 18, 19, or 20 amino acids may be identical to those in the corresponding PDE4 sequence.

Without wishing to be bound by any particular theory, it is believed that residues X470, X471, X477, X481. X482, X485 and X492 are particularly significant in the interaction between PDE4 and HSP20. It is not necessary for all of these residues to be present in order for binding to take place. However, when present, it may be desirable that individually X470 is H, X471 is H, X477 is K, X481 is E, X482 is E, X485 is D and/or X492 is K.

The binding moiety may comprise the sequence

### HHLAVGFKLLQEENCDIFQNLTK (SEQ ID NO: 33)

or a fragment thereof, e.g. a fragment of at least 2, 3, 4 or 5 amino acids, capable of binding to HSP20. In particular, the HSP20 binding moiety may comprise a C-terminal K residue. The binding moiety may further comprise up to 11 substitutions or deletions relative to this native PDE4 sequence. The binding moiety may comprise up to 1, 2, 3, 4, 5, 6, 7, 8, 9 or 10 substitutions or deletions relative to the native PDE4 sequence.

In preferred embodiments, the HSP20 binding moiety may comprise a C-terminal X491-X492 sequence, wherein X491 is T, S, A or G, and X492 is K. In some embodiments, the C-terminal sequence comprises FQNLTK (SEQ ID NO: 4) or LTK. Optionally, F may be substituted with any one of M, I, L, G or A; Q may be substituted with N, G or A; N may be substituted with Q, G or A; L may be substituted with I, M, A or G; and T may be substituted with T, A or G. Optionally, the HSP20 binding moiety may consist or consist essentially of the C-terminal sequence described above.

An antagonist as described herein may comprise one or more heterologous components. A heterologous component is typically a moiety with a function other than binding to HSP20. Where the heterologous component is protein in nature, it typically is not derived from PDE4, i.e. it has a sequence which does not have more than 25% sequence identity with any stretch of PDE4 sequence of the same length.

The heterologous component may modulate a property of the antagonist such as stability, activity, immunogenicity, solubility, bioavailability, membrane permeability or localisation. For example, the heterologous component may be used to increase or reduce half-life in vitro or in vivo.

Thus the antagonist may be chemically derivatised with a heterologous component in order to modify its pharmacokinetic and/or activity properties. For example, the heterologous component may be a non-proteinaceous molecule chemically linked to the antagonist. Examples include polyethylene glycol (PEG), poly-sialic acid and fatty acyl moieties such as stearate.

The heterologous component may be a protein or domain thereof. For example, it may be an immunoglobulin Fc region. Fusions between Fc regions and non-immunoglobulin components are often referred to as immunoadhesins. Alternatively it may be a protein such as albumin which can be used to extend half-life in vivo.

The heterologous component may be a moiety to increase trans-membrane transport of the antagonist. For example, the antagonist may be acylated with a suitable fatty acid such as a stearoyl group, or comprise a peptoid moiety. Alternatively it may comprise a transducing peptide sequence (“peptide transduction domain”, or PTD),

The heterologous component may be cleavable from the antagonist. For example, the heterologous component may be cleaved before or after the antagonist enters a cell.

The heterologous component may prevent the antagonist from inhibiting PDE4 binding to HSP20. An antagonist having such a heterologous component may be an inactive “pro-drug”, which is activated when the heterologous component is modified e.g. cleaved before or after the antagonist enters a cell. Thus the invention further provides a pro-drug form of an antagonist of the invention: wherein the pro-drug form is activated when a heterologous component is modified e.g. cleaved before or after the antagonist enters a cell.

Where appropriate, the heterologous component may be expressed as a fusion protein with the HSP20-binding moiety.

In the case of fusion proteins, a flexible peptide linker is typically included between the two components to allow the two components to interact freely with one another without steric hindrance. The skilled person is perfectly capable of designing a suitable linker. Conventionally, such linkers are between 12 and 20 amino acids in length, and have a high proportion of small and hydrophilic amino acid residues (e.g. glycine and serine) to provide the required flexibility without compromising aqueous solubility of the molecule. The linker sequence may comprise a cleavage signal sequence.

The antagonist may be modified or derivatised at the N- and/or C-terminus, for example to improve stability (e.g. against proteolysis), to improve interaction with HSP20, or for other purposes. For example, the N-terminus may be alkylated (e.g. with a C1-4 alkyl group, such as a methyl group) or acylated (e.g. with an acetyl, formyl, benozyl or trifluoroacetyl group). The C-terminus may be amidated. The N-terminus may be glycosylated or otherwise derivatised, e.g. by conjugation of polyethylene glycol to the antagonist (commonly referred to as PEGylation). The invention further provides that the N-terminus may comprise a capping group. For example, the N-terminus may comprise a capping group formed by a condensation reaction with any one of the following:

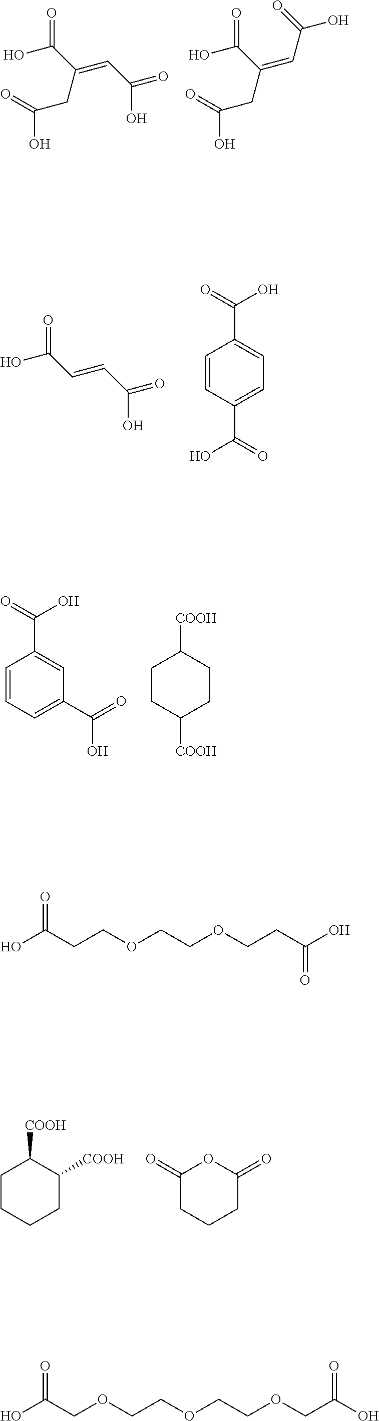

Methods of the invention may be applied in any appropriate biological system, whether in vivo, ex vivo or in vitro. In general, the system will contain HSP20. The system may also contain a PDE4, e.g. PDE4D5.

An in vitro biological system may comprise an isolated sample of tissue, blood, plasma or serum, or an isolated cell. A tissue sample may be, for example, from brain, heart, skin or scar tissue, or any tissue in which HSP20 is expressed. Suitable cell types include cells from the brain, heart or skin, neuronal cells, cardiomyocytes, fibroblasts, or any cell type where HSP20 is expressed endogenously or ectopically.

Alternatively, the system may be assembled in vitro from individual components such as isolated proteins (e.g. recombinant proteins), cells (which may be isolated from tissue, or grown in culture), etc.

The methods of the invention may also be applied in vivo in situations where increased HSP20 activation is desirable. Such situations include pathological conditions in which HSP20 activation has a protective or therapeutic effect, on the pathogenesis or symptoms of the condition, either directly or indirectly.

Conditions in which activation of HSP20 has been identified to have a beneficial effect on pathogenesis or symptoms include cerebral amyloid angiopathy, Alzheimer's disease, forebrain ischemia, cardiac disease, cardiac ischemia/reperfusion, chronic beta-adrenergic stimulation, pharmacological treatment by doxorubicin, vasospasm and platelet aggregation, endotoxin induced myocardial dysfunction, congestive heart failure, ischemia/reperfusion injury, apoptosis, cell necrosis, scar tissue formation, and fibrotic disorders, interstitial fibrosis and apoptosis arising from constant β-agonist treatment, and delayed decreases in cerebral perfusion following subarachnoid hemorrhage. However, any condition in which HSP20 activation has a positive effect on the health of the individual may be treated by the products and methods described herein. In particular, products and methods of the invention may be useful in conditions under which cells are physiologically stressed.

Thus the invention provides a method of treating a pathological condition as described above in an individual, comprising administering an antagonist capable of inhibiting PDE4 binding to HSP20 to said individual.

The invention also provides an antagonist capable of inhibiting PDE4 binding to HSP20, for use in a method of medical treatment, for example for treating a pathological condition as described above.

The invention also provides use of an antagonist capable of inhibiting PDE4 binding to HSP20 in the preparation of a medicament for the treatment of a pathological condition as described above.

The antagonist may be any antagonist capable of inhibiting PDE4 binding to HSP20 described herein.

The invention further provides a method of prophylactic treatment of a suitable pathological condition in an individual, comprising administering an antagonist capable of inhibiting PDE4 binding to HSP20 to said individual.

The invention also provides methods by which agents may be screened for an ability to inhibit the interaction between HSP20 and PDE4. Agents screened by the methods of the invention may have utility in the methods of the invention, for example therapeutic utility in the treatment or prophylaxis of pathological conditions, as described herein.

Broadly, then, the invention provides a method of screening for an agent capable of inhibiting binding between PDE4 and HSP20, the method comprising providing a candidate agent; and testing the candidate agent for the ability to inhibit binding between PDE4 or a fragment thereof and HSP20 or a fragment thereof.

Various methods will be apparent by which such testing can be performed.

In one embodiment the invention provides a method of testing a candidate agent for an ability to inhibit binding between PDE4 and HSP20, comprising contacting HSP20 or a fragment thereof capable of binding to PDE4 with

(i) PDE4 or a fragment thereof capable of binding to HSP20; and
(ii) the candidate agent;
and determining the binding of PDE4 or fragment thereof with said HSP20 or fragment thereof.

Typically, the method will involve the steps of determining binding between the HSP20 and PDE4 (or fragments thereof as applicable) in the presence and the absence of the candidate agent. A decreased level of binding in the presence of the candidate agent, as compared to the level of binding seen in the presence of the candidate agent, indicates that the candidate agent is capable of inhibiting binding between PDE4 and HSP20.

A candidate agent may inhibit binding between PDE4 and HSP20 by binding HSP20 or a fragment thereof, or by binding PDE4 or a fragment thereof. For example, a candidate agent may be a fragment of HSP20 capable of binding PDE4.

In an alternative embodiment, the invention provides a method of screening for an agent capable of inhibiting binding between PDE4 and HSP20, the method comprising

(i) providing a candidate agent; and
(ii) testing the candidate agent for the ability to inhibit binding between PDE4 or a fragment thereof and HSP20 or a fragment thereof.

In an alternative embodiment, the invention provides a method of testing a candidate agent for an ability to inhibit binding between PDE4 and HSP20, comprising contacting the candidate agent with

(i) HSP20 or a fragment thereof; and
(ii) an HSP20-binding moiety as defined above which is capable of binding to HSP20 or said fragment thereof and of inhibiting binding of PDE4 to HSP20; and determining binding between (i) and (ii).

This version of the method will typically involve the steps of determining binding between (i) and (i) in the presence and the absence of the candidate agent. A decreased level of binding in the presence of the candidate agent, as compared to the level of binding seen in the presence of the candidate agent, indicates that the candidate agent is capable of inhibiting binding between (i) and (ii) and is therefore likely to inhibit binding between PDE4 and HSP20.

In other words, the above methods provide a method of testing a candidate agent for an ability to increase activation of HSP20 (i.e. increase phosphorylation of HSP20 on Ser16), comprising the steps outline above.

The invention further provides a method of optimising an HSP20-binding moiety for the ability to inhibit binding between PDE4 and HSP20, comprising

(i) providing a parent HSP20-binding moeity capable of inhibiting binding between PDE4 and HSP20,
(ii) making a variant of the parent HSP20-binding moiety,
(iii) testing the variant for the ability to inhibit binding between PDE4 and HSP20; and optionally
(iv) comparing the ability of the variant to inhibit binding between PDE4 and HSP20 to the ability of the parent HSP20-binding moiety.

The comparison may be direct. For example, the assay may be performed in a competitive format as set out above, in which both the parent and variant are contacted simultaneously with HSP20.

Alternatively the ability of the variant polypeptide may be compared to a pre-determined level of binding for the parent HSP20-binding moiety. The pre-determined value will typically have been determined under comparable or identical conditions.

The parent HSP20 binding moiety may be a protein, and the variant produced by modification (e.g. substitution, deletion or addition) of one or more amino acids compared to the parent sequence.

In any of the screening methods described herein, the candidate agent may be a small molecule, peptide, polypeptide, peptidomimetic, or any other suitable compound.

For the purposes of the testing or screening methods described herein, either:

(i) the HSP20 or fragment thereof and PDE4 or fragment thereof form a specific binding pair which interact specifically with one another; or
(ii) the HSP20 or fragment thereof and HSP20-binding moiety form a specific binding pair which interact specifically with one another.

Either member of the binding pair may be immobilised on a solid phase, and contacted with a sample containing the other member of the binding pair. The candidate agent may then be introduced before, concurrently with, or after the sample containing the member of the binding pair which is not immobilised on a solid phase.

Whether or not a member of the binding pair is immobilised, one or both members, or another component of the assay, may be labelled in order to facilitate detection of binding.

For the purposes of the testing or screening methods, a PDE4 fragment is a portion of a full length PDE4 amino acid sequence (or of a sequence having up to at least 80% identity therewith) which is capable of binding HSP20.

An HSP20 fragment is a portion of the full length HSP20 amino acid sequence (or of a sequence having up to at least 80% sequence identity therewith) capable of binding PDE4.

Any suitable format may be used for the assay, including high-throughput formats. The skilled person is aware of numerous suitable formats for such screening assays, and will be capable of selecting an appropriate format depending on their individual requirements.

In a further aspect, the invention provides an antagonist capable of inhibiting binding between PDE4 and HSP20 as described herein. In one embodiment, the antagonist comprises an HSP20-binding moiety as described herein, with the proviso that the antagonist does not consist of amino acid residues 461 to 485, 466 to 490, 471 to 495 or 476 to 500 of PDE4D5, or a full length PDE4 protein.

The antagonist may consist of an HSP20-binding moiety as described herein, with the proviso that the antagonist does not consist of amino acid residues 461 to 485, 466 to 490, 471 to 495 or 476 to 500 of PDE4D5, or a full length PDE4 protein.

An antagonist of the invention may be used in any of the methods of the invention described herein as appropriate.

The invention further provides an isolated nucleic acid encoding an antagonist of the invention.

The invention further provides a vector (e.g. an expression vector) comprising a nucleic acid of the invention.

The invention also provides a host cell comprising a nucleic acid or a vector of the invention. The host cell may be prokaryotic or eukaryotic as desired.

The invention further provides a composition comprising an antagonist, or a salt or derivative thereof, a nucleic acid, vector or host cell of the invention and a carrier, in preferred embodiments, the composition is a pharmaceutically acceptable composition and the carrier is a pharmaceutically acceptable carrier.

As already described, the invention extends to expression vectors comprising the above-described nucleic acid sequence, optionally in combination with sequences to direct its expression, and host cells transformed with the expression vectors. Preferably the host cells are capable of expressing and secreting the antagonist of the invention. In a still further aspect, the present invention provides a method of producing the antagonist of the invention, the method comprising culturing the host cells under conditions suitable for expressing the antagonist and purifying the antagonist thus produced.

The invention further provides a nucleic acid of the invention, an expression vector of the invention, or a host cell capable of expressing and secreting an antagonist of the invention, for use in therapy. It will be understood that the nucleic acid, expression vector and host cells may be used for treatment of any of the disorders described herein that may be treated with an antagonist of the invention itself. References to a composition comprising an antagonist of the invention, or administration of an antagonist of the invention, should therefore be construed to encompass administration of a nucleic acid, expression vector or host cell of the invention except where the context demands otherwise.

In a further aspect, the invention provides a method of determining whether a patient will respond to treatment with an antagonist of the invention, comprising the steps of

(i) isolating a sample from a patient,
(ii) contacting the sample with an antagonist of the invention,
(iii) comparing HSP20 activation between (i) and (ii), wherein an increase in HSP20 activation is a positive indicator that the patient will response to treatment.

The invention will now be described in more detail, by way of example and not limitation, by reference to the accompanying drawings. Many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

### DESCRIPTION OF THE FIGURES

FIG. 1. The phosphorylation of HSP20 at Ser16 is modulated by PDE4 activity in HEKB2 cells. A, HEKB2 cells overexpressing HSP20 or HSP20 ser16-Ala mutant were treated with isoprenaline for indicated times. Cellular lysates were blotted for HSP20 and Phospho-Ser16 HSP20. B, HEKB2 cells overexpressing HSP20 were treated with isoprenaline for indicated times, Cellular lysates were blotted for HSP20 and Phospho-Ser16 HSP20. C, HEKB2 cells overexpressing HSP20 were treated with isoprenaline for indicated times following pre-treatment with the PDE4-specific inhibitor, rolipram. Cellular lysates were blotted for HSP20 and Phospho-Ser16 HSP20. D, Quantifications of data from FIGS. 1B and 1C (n=4). * Denotes significance at a level p<0.05 E, Lysate from untransfected HEKB2 cells was blotted for HSP20 and Phospho-Ser16 HSP20.

FIG. 2. The phosphorylation of HSP20 at Ser16 is modulated by PDE4 activity in cardiac myocytes. A, Neonatal cardiac myocytes were treated with isoprenaline for indicated times with or without pre-treatment with the PDE4-specific inhibitor, rolipram. Cellular lysates were blotted for HSP20 and Phospho-Ser16 HSP20. B, Quantifications of data from FIG. 2A (n=3). * Denotes significance at a level p<0.05 over iso treatment.

FIG. 3. HSP20 forms a complex with members of the PDE4 family of phosphodiesterases. A (upper panel), Immunopurification (Ips) of HSP20 from cellular lysates isolated from myocytes following treatment with Isoprenaline (1 μM) were blotted for the presence of PDE4D isoforms. B, (lower panel) The PDE4 activity associated with HSP20 Ips was determined. C, Members of the PDE4D sub-family of PDE4s were co-expressed with HSP20 in HEKB2 cells. HSP20 Ips were probed for PDE4D isoforms.

FIG. 4, Mapping the site of HSP20 interaction on the catalytic unit of PDE4. A, Peptide array technology was used to screen the complete sequence of PDE4D5 (SEQ ID NO: 35) for areas of HSP20-His association. The initial screen identified four 25mer peptide sequences covering V⁴⁵⁶-L⁴⁹⁰ (spots 93, 94, 95 and 96) that interacted with HSP20. This area was subjected to alanine scanning analysis where every residue between E⁴⁶⁸ and K⁴⁹² was sequentially substituted by an alanine residue and the overlay of HSP20-His was repeated. B, PDE4D5 residues crucial for the interaction of HSP20 on peptide array were superimposed on the crystal structure of the PDE4D catalytic unit.

FIG. 5. Measurement of relative cAMP concentration in the vicinity of HSP20 using targeted cAMP FRET probes. A, Representative traces showing FRET ratios that indicate relative cAMP concentrations measured by cytosolic and HSP20 targeted cAMP reporter constructs. B, Upper panel, graphical depiction of maximum FRET ratios achieved using both cytosolic and HSP20-targeted probes following treatment with isoprenaline (Iso), rolipram (Roli) and IBMX. Lower panel, images of the localisation of cytosolic and HSP20-targeted cAMP reporter constructs.

FIG. 6. Peptide disruption of the PDE4D-HSP20 complex promotes PKA phosphorylation on Ser16. A, Upper panel. HEK293 cells were transfected with HSP20 and PDE4D5. Immunoprecipitation of HSP20 done in the presence increasing concentrations of Peptide 906 (SEQ ID NO: 36) and Peptide control (SEQ ID NO: 37) were probed for PDE4D5. Lower panel. Densitometric analysis of amount of PDE4D5 in HSP20 immunoprecipitates normalised to expression level. Data representative of n=3. B, Purified preparations of HSP20-His and PDE4D5-GST were subject to an ELISA assay containing increasing amounts of ‘Peptide 906’ and ‘peptide control’. C, Upper panel, HSP20 was transfected into HEK293 cells and treated with ‘Peptide 906’ for indicated times. Cellular lysates were blotted for HSP20 and phospho-HSP20. Middle panel, HSP20 was transfected into HEK293 cells and treated with ‘Peptide 906’ or ‘peptide control’ for indicated times. Cellular lysates were blotted for HSP20 and phospho-HSP20. Lower panel, Densitometric analysis of phosphorylation of HSP20 normalised to expression level (n=3).

FIG. 7. Peptide disruption of the PDE4D-HSP20 complex attenuates the hypertrophic response triggered by constant □-agonist treatment. A, Upper panel. The area covered by isolated cardiac myocytes was used as a measure of cell size. Lower panel, Graphical depiction of the mean areas (N=100) of cells following 24 hours of indicated treatments. B, impedance measurements were used to calculate continuous normalised cell index using exCELLigence technology Normalised mean (n=4) cell index after 24 hours of indicated treatments.

FIG. 8. Identification of shorter HSP20-binding peptides (SEQ ID NOs: 36, 38-67)

A variety of peptides were generated and SPOT-synthesised onto slides which were then overlaid with 10 μg/ml of purified His-Hsp20. Interaction with peptides and Hsp20 was evaluated by probing slides with anti-His or Hsp20 antibodies and analysed using enhanced chemi-luminescence techniques, Negative controls used anti-His antibody only with no overlay of His-Hsp20 protein (data not shown). Positive interaction domains were then subjected to further rounds of truncations and key endogenous residues were substituted for non-native residues to ascertain what effects this would have in the binding of Hsp20.

FIG. 9. Disrupter induced HSP20 phosphorylation SH-SY5Y cells nucleofected with 500 μM of each peptide and incubated for 2 hours prior to harvesting. Each assay was blotted for Hsp20, pS16-Hsp20 and tubulin. Changes in levels of phospho-Hsp20 relative to DMSO only controls were quantified through densitometry analysis and each assay was normalised to α-tubulin loading control. The results are the average of three separate experiments and error bars represent SEM. *=p<0.05, **=p<0.01 compared to DMSO only controls (Student-T-test)

### DETAILED DESCRIPTION OF THE INVENTION

Throughout this specification, the conventional one-letter and three-letter codes for natural amino acids are used as well as generally accepted three letter codes for other α-amino acids. All amino acid residues in peptides of the invention are preferably of the L-configuration; however, D-configuration amino acids may also be present. Examples of α-amino acids include both natural amino acids and non-natural amino acids.

The natural amino acids include: those with nonpolar (i.e. hydrophobic) side chains (“R groups”):

alanine, Ala, A; glycine, Gly, G; isoleucine, Ile, I; leucine, Leu, L; methionine, Met, M; phenylalanine, Phe, F; proline, Pro, P; tryptophan, Trp, W; and valine, Val, V;
those with polar but uncharged R groups:
asparagine, Asn, N; cysteine, Cys, C; glutamine, Gln, Q; serine, Ser, S; threonine, Thr, T; and tyrosine, Tyr, Y;
those with potentially positively charged (or basic) side chains:
arginine, Arg, R; histidine, His, H; and lysine, Lys, K;
those with potentially negatively charged (or acidic) side chains: aspartic acid, Asp, D; glutamic acid, Glu, E.

Examples of non-natural α-amino acids include: those with nonpolar (i.e. hydrophobic) side chains (“R groups”):

sarcosine, norleucine, α-aminoisobutyric acid (Aib); and
those with potentially positively charged (or basic) side chains:
ornithine, 2,4-diaminobutyric acid and 2,3-diaminopropanoic acid (Dpr).

Non-naturally occurring analogues of glutamate include:

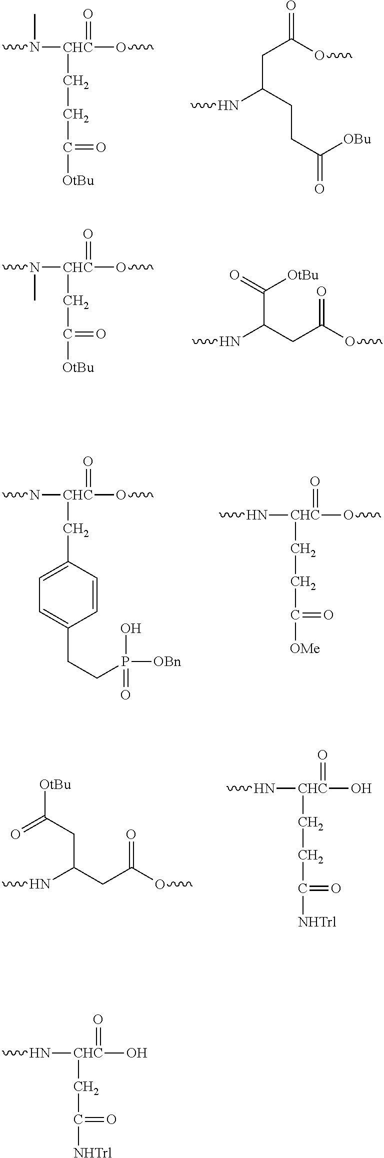

These non-naturally occurring analogues may also be used as substitutes for other amino acids with potentially negatively charged side chains.

Non-naturally occurring analogues of phenylalanine include:

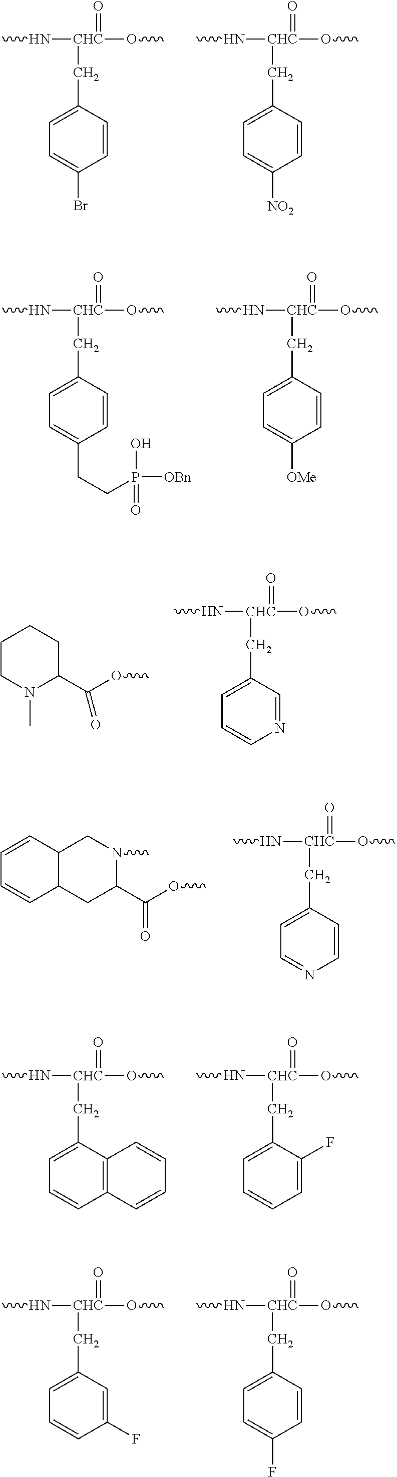

These non-naturally occurring analogues may also be used as substitutes for other amino acids with non-polar side chains.

Non-naturally occurring analogues of glycine include:

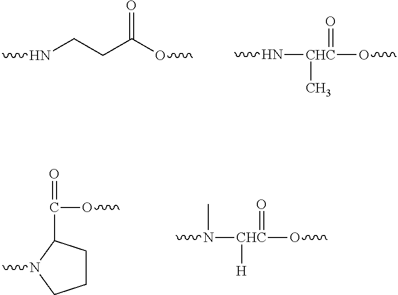

These non-naturally occurring analogues may also be used as substitutes for other amino acids with non-polar side chains.

Antagonists of the invention may additionally comprise amino acids with chemical modification of one or more of its amino acid side groups, α-carbon atoms, terminal amino group, or terminal carboxylic acid group. A chemical modification includes, but is not limited to, adding chemical moieties, creating new bonds, and removing chemical moieties. Modifications at amino acid side groups include, without limitation, acylation of lysine ε-amino groups, N-alkylation of arginine, histidine, or lysine, alkylation of glutamic or aspartic carboxylic acid groups, and deamidation of glutamine or asparagine. Examples of modified natural amino acids include, but are not limited to, hydroxyproline, γ-carboxyglutamate, and O-phosphoserine.

Amino acids may also be chemically modified with protecting groups that protect certain chemical groups during peptide synthesis. For example, amino, carboxylic acid or phosphate groups may be protected during peptide synthesis to prevent inappropriate reactions, and these protecting groups may be removed during peptide synthesis or after synthesis is complete, as appropriate. For example, 9-fluorenylmethyloxycarbonyl (Fmoc) or tert-butoxycarbonyl (t-Boc) may be used during solid-phase peptide synthesis to protect the α-amino group of amino acid monomers.

Amino acid “R” group side chains may also be protected with protecting groups during peptide synthesis. For example, protecting groups that may be used include: Methyl, Formyl, Ethyl, Acetyl, t-Butyl, Anisyl, Benzyl, Trifluoroacetyl, N-hydroxysuccinimide, t-Butyloxycarbonyl, Benzoyl, 4-Methylbenzyl, Thioanizyl, Thiocresyl, Benzyloxymethyl, 4-Nitrophenyl, Benzyloxycarbonyl, 2-Nitrobenzoyl, 2-Nitrophenylsulphenyl, 4-Toluenesulphonyl, Pentafluorophenyl, Diphenylmethyl, 2-Chlorobenzyloxycarbonyl, 2,4,5-trichlorophenyl, 2-bromobenzyloxycarbonyl, 9-Fluorenylmethyloxycarbonyl, Triphenylmethyl, and 2,2,5,7,8-pentamethyl-chroman-6-sulphonyl.

Optionally, antagonists of the invention may comprise amino acids modified with one or more protecting groups.

Optionally, a protecting group described herein may be used as an N-terminal capping group.

It should be understood that an antagonist of the invention might also be provided in the form of a salt or other derivative. Salts include pharmaceutically acceptable salts such as acid addition salts and basic salts. Examples of acid addition salts include hydrochloride salts, citrate salts and acetate salts. Examples of basic salts include salts where the cation is selected from alkali metals, such as sodium and potassium, alkaline earth metals, such as calcium, and ammonium ions ⁺N(R³)₃(R⁴), where R³ and R⁴ independently designates optionally substituted C₁₋₆-alkyl, optionally substituted C₂₋₆-alkenyl, optionally substituted aryl, or optionally substituted heteroaryl. Other examples of pharmaceutically acceptable salts are described in “Remington's Pharmaceutical Sciences”, 17th edition. Ed. Alfonso R. Gennaro (Ed.), Mark Publishing Company, Easton, Pa., U.S.A., 1985 and more recent editions, and in the Encyclopaedia of Pharmaceutical Technology.

Other derivatives of an antagonist of the invention include coordination complexes with metal ions such as Mn²⁺ and Zn²⁺, esters such as in vivo hydrolysable esters, free acids or bases, hydrates, prodrugs or lipids. Esters can be formed between hydroxyl or carboxylic acid groups present in the antagonist and an appropriate carboxylic acid or alcohol reaction partner, using techniques well known in the art. Derivatives which as prodrugs of the antagonist are convertible in vivo or in vitro into one of the parent antagonists. Typically, at least one of the biological activities of the antagonist will be reduced in the prodrug form of the antagonist, and can be activated by conversion of the prodrug to release the antagonist or a metabolite of it. Examples of prodrugs include the use of protecting groups which may be removed in situ releasing active antagonist or serve to inhibit clearance of the antagonist in vivo.

An antagonist of the invention may be any chemical agent capable of inhibiting the binding, interaction or association between HSP20 and PDE4. An antagonist of the invention may comprise, for example, a peptide, polypeptide, small molecule, peptidomimetic, antibody, antibody fragment, antibody Fc region, immunoadhesin, or fusion protein, or any combination of chemical agents.

The antagonist comprises or consists of an HSP20-binding moiety, which is capable of binding to HSP20 and inhibiting interaction with PDE4, i.e. it is capable of inhibiting binding of PDE4 to HSP20.

The HSP20-binding moiety may comprise or consist of an antibody binding domain specific for HSP20 and capable of inhibiting binding of PDE4 to HSP20.

It may alternatively comprise or consist of a fragment of PDE4 capable of binding to HSP20 or an analogue thereof. In certain embodiment, the HSP20-binding moiety comprises at least 2, 3, 4, 5, 6, 7, 8, 9, 10, 15, 20, 25, 30, 35, 40, 45, 50 or more consecutive amino acids of PDE4. In some embodiments, the HSP20-binding moiety has a maximum of 2, 3, 4, 5, 6, 7, 8, 9, 10, 11, 12, 13, 14, 15, 20, 25, 30, 35, 40, 45, 50, or 100 consecutive amino acids of PDE4. In a further embodiment, the HSP20-binding moiety is a maximum of 2, 3, 4, 5, 6, 7, 8, 9, 10, 11, 12, 13, 13, 15, 20, 25, 30, 35, 40, 45, 50, or 100 amino acids in length,

### PDE4

Over 20 different isoforms of PDE4 exist in humans, via splicing of 4 PDE4 genes (A, B, C, D), which share a highly conserved catalytic domain. PDE4 homologues exist in all eukaryotic species, and are highly conserved between mammals. The sequence of one isoform, human PDE4D5, is as follows (SEQ ID NO: 68):

| | |
|---|---|
| 1 | MAQQTSPDTL TVPEVDNPHC PNPWLNEDLV KSLRENLLQH |
| | EKSKTARKSV SPKLSPVISP |
| 61 | RNSPRLLRRM LLSSNIPKQR RFTVAHTCFD VDNGTSAGRS |
| | PLDPMTSPGS GLILQANFVH |
| 121 | SQRRESFLYR SDSDYDLSPK SMSRNSSIAS DIHGDDLIVT |
| | PFAQVLASLR TVRNNFAALT |
| 181 | NLQDRAPSKR SPMCNQPSIN KATITEEAYQ KLASETLEEL |
| | DWCLDQLETL QTRHSVSEMA |
| 241 | SNKFKRMLNR ELTHLSEMSR SGNQVSEFIS NTFLDKQHEV |
| | EIPSPTQKEK EKKKRPMSQI |
| 301 | SGVKKLMHSS SLTNSSIPRF GVKTEQEDVL AKELEDVNKW |
| | GLHVFRIAEL SGNRPLTVIM |
| 361 | HTIFQERDLL KTFKIPVDTL ITYLMTLEDH YHADVAYHNN |
| | IHAADVVQST HVLLSTPALE |
| 421 | AVFTDLEILA AIFASAIHDV DHPGVSNQFL INTNSELALM |
| | YNDSSVLENH FILAVGFKLLQ |
| 481 | EENCDIFQNL TKKQRQSLRK MVIDIVLATD MSKHMNLLAD |
| | LKTMVETKKV TSSGVLLLDN |
| 541 | YSDRIQVLQN MVHCADLSNP TKPLQLYRQW TDRIMEEFFR |
| | QGDRERERGM EISPMCDKHN |
| 601 | ASVEKSQVGF IDYIVHPLWE TWADLVHPDA QDILDTLEDN |
| | REWYQSTIPQ SPSPAPDDPE |
| 661 | EGRQGQTEKF QFELTLEEDG ESDTEKDSGS QVEEDTSCSD |
| | SKTLCTQDSE STEIPLDEQV |
| 721 | EEEAVGEEEE SQPEACVIDD RSPDT |

In this specification, “PDE4” means the polypeptide sequence of any PDE4 isoform or homologue capable of binding HSP20. Likewise, a “PDE4 fragment” means a partial polypeptide sequence of any PDE4 isoform or homologue capable of binding HSP20.

### HSP20

HSP20, also known as HspB6, is one of at least 10 known small heat shock proteins. HSP20 has been shown to have a therapeutic effect in a range of diseases, described herein, and a key facet of the protective mechanism of HSP20 relates to its phosphorylation on Ser 6. The sequence of human HSP20 is as follows (SEQ ID NO: 69):

| | |
|---|---|
| 1 | MEIPVPVQPS WLRRASAPLP GLSAPGRLFD QRFGEGLLEA |
| | ELAALCPTTL APYYLRAPSV |
| 61 | ALPVAQVPTD PGHFSVLLDV KHFSPEEIAV KVVGEHVEVH |
| | ARHEERPDEH GFVAREFHRR |
| 121 | YRLPPGVDPA AVTSALSPEG VLSIQAAPAS AQAPPPAAAK |

In this specification, “HSP20” means the polypeptide sequence of HSP20 or that of a homologue. An “HSP20 fragment” means a partial polypeptide sequence of HSP20 or that of a homologue, capable of binding PDE4.

### Screening and Other Assay Methods

The invention provides methods of screening for compounds capable of inhibiting the interaction between PDE4 and HSP20.

Interactions between PDE4 and HSP20 (or fragment) may be studied in vitro by immobilising one on a solid support and bringing the other into contact with it.

The immobilised protein is generally contacted with a sample containing the other protein under appropriate conditions which allow the two proteins to bind to one another (or would allow such binding in the absence of any inhibitor or candidate inhibitor). The fractional occupancy of the binding sites on the immobilised component can then be determined either directly or indirectly, e.g. by labelling the component in the sample or by using a developing agent or agents to arrive at an indication of the presence or amount of the component in the sample.

Typically, the developing agents are directly or indirectly labelled (e.g. with radioactive, fluorescent or enzyme labels, such as horseradish peroxidase) so that they can be detected using techniques well known in the art. Directly labelled developing agents have a label associated with or coupled to the agent. Indirectly labelled developing agents may be capable of binding to a labelled species (e.g. a labelled antibody capable of binding to the developing agent) or may act on a further species to produce a detectable result. Thus, radioactive labels can be detected using a scintillation counter or other radiation counting device, fluorescent labels using a laser and confocal microscope, and enzyme labels by the action of an enzyme label on a substrate, typically to produce a colour change. In further embodiments, the developing agent or analyte may be tagged to allow its detection, e.g. linked to a nucleotide sequence which can be amplified in a PCR reaction. The developing agent(s) can be used in a competitive method in which the developing agent competes with the analyte for occupied binding sites of the binding agent, or non-competitive method, in which the labelled developing agent binds analyte bound by the binding agent or to occupied binding sites. Both methods provide an indication of the number of the binding sites occupied by the analyte, and hence the concentration of the analyte in the sample, e.g. by comparison with standards obtained using samples containing known concentrations of the analyte.

Preferred assay formats include immunological techniques such as ELISA assays.

Alternatively, techniques such as surface plasmon resonance may be used to monitor binding between the two proteins directly, without the need for either component to be labelled.

The protein which is immobilized may be immobilized using an antibody against that protein bound to a solid support or via other technologies which are known per se, including simply coating the protein on a suitable surface, such as a well of a microtiter plate. A preferred in vitro interaction may utilise a fusion protein including glutathione-S-transferase (GST), which may be immobilized on glutathione agarose beads.

There is also an increasing tendency in the diagnostic field towards miniaturisation of such assays, e.g. making use of binding agents (such as antibodies or nucleic acid sequences) immobilised in small, discrete locations (microspots) and/or as arrays on solid supports or on diagnostic chips. These approaches can be particularly valuable as they can provide great sensitivity (particularly through the use of fluorescent labelled reagents), require only very small amounts of biological sample from individuals being tested and allow a variety of separate assays can be carried out simultaneously. This latter advantage can be useful as it provides an assay employing a plurality of analytes to be carried out using a single sample.

As already described, such assay methods may be used to screen for compounds capable of inhibiting binding between HSP20 and PDE4. Candidate agents identified by such screens may be subjected to one or more rounds of modification and re-testing in order to identify further agents having improved properties. The skilled person will be aware of numerous suitable screening methods and will be able to design appropriate protocols for identification of candidate binding agents.

### Antibodies

It has been shown that fragments of a whole antibody can perform the function of binding antigens. The term “antibody” is therefore used herein to encompass any molecule comprising the binding fragment of an antibody, Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding member (Bird et al, Science, 242, 423-426, 1988; Huston et al, PNAS USA, 85, 5879-5883, 1988). The skilled person is perfectly aware of methods of generating antibodies against a target molecule, which are known in the art.

The invention provides an antibody capable of inhibiting binding between HSP20 and PDE4. An antibody of the invention may bind either HSP20 or PDE4. In one embodiment, an antibody recognizes an epitope within a fragment of PDE4 capable of binding HSP20. In a further embodiment, an antibody recognizes an epitope within a fragment of HSP20 capable of binding PDPE4. An antibody of the invention could therefore be used in any of the methods of the invention discussed herein.

### Peptoids

An antagonist of the invention may comprise a peptoid moiety which may, for example, be used to increase trans-membrane transport of the antagonist. Peptoids (or poly-N-substituted glycines) have side chains that are connected to the nitrogen atom of the peptide backbone, rather than to the α-carbon as in amino acids. Peptoids have been used previously to increase cell permeability, as described in Peretto, et at Chem Commun, 2003, 18, 2312-3 and Unciti-Broceta et al, Bioorg Med Chem, 2009, 17, 959-66.

### Pharmaceutical Compositions

The antagonists and other therapeutic agents described in this specification can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes or topical application.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride injection, Ringer's Injection, Lactated Ringer's injection. Preservatives, stabilisers, buffers, antioxidants and/or other additives may be included, as required.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Suitable carriers, adjuvants, excipients, etc. can be found in standard pharmaceutical texts, for example Remington's Pharmaceutical Sciences, 20th Edition, 2000, pub. Lippincott, Williams & Wilkins; and Handbook of Pharmaceutical Excipients, 2nd edition, 1994.

### Combination Therapy

The antagonists and other therapeutic agents described herein may be administered as part of a combination therapy with another agent for treatment of a given pathological condition.

### EXAMPLES

### Materials and Methods

PolyFect transfection reagent was from Qiagen. Proteases inhibitor cocktail tablets were from Roche. Anti-HSP20 antibody was from Millipore (07-490), Anti-VSV antibody was from Abcam (ab1874), Anti-phospho-HSP20 antibody was from Abcam (ab58522). Isoprenaline were from Sigma, Protein G beads were from Amersham. ECL was from Pierce. Peptide 906 and peptide control were synthesized, dissolved in DMSO to a stock concentration of 10 mM and used at a final concentration of 10 μM.

### Generation of Hsp20 Expression Constructs

Ultimate™ ORF clone IOH57317 (Invitrogen), containing the open reading frame of Hsp20 in pENTR221 vector, was used to generate pcDNA6.2-Hsp20, by Gateway cloning into pcDNA6.2/V5-DEST (invitrogen).

Generation of Hsp20-Targeted cAMP FRET Sensor

The Hsp20-targeted cAMP FRET probe was generated by cloning the Hsp20 open reading frame, excluding the stop codon, immediately 5′ to the EPACG-based FRET sensor⁵⁵. Hsp20 open reading frame was amplified from the Ultimate™ ORF clone IOH57317 (Invitrogen) using the following primers to incorporate HindIII restriction sites at both 5′ and 3′ ends: Forward 5′-gcacaagcttATGGAGATCCCTGTGCCTGTGC-3′ (SEQ ID NO: 70) Reverse 5′-gcacaagcttCTTGGCTGCGGCTGGCGGTGG-3′ (SEQ ID NO: 71). The resulting fragment was cloned, in frame, into the HindIII site 5′ to the EPAC1-based sensor, clones were screened for correct orientation of insert and correct clones were sequenced.

### Site Directed Mutagenesis

HSP20 mutants were constructed using QuickChange (Stratagene) and verified by DNA sequencing.

### Cell Culture, Transfection, Immunoprecipitation and Western Blotting.

HEK293B2 cells are a stable cell line overexpressing the GFP-tagged β₂AR were cultured as previously described⁴⁸. HEK293B2 cells were transiently transfected with wild type HSP20 or a phosphorylation defective HSP20 (S16A) or a phosphorylation mimic HSP20 (S16D). Neonatal cardiac myocytes were prepared and cultured as previously described³⁰. Cell lysis, immunopurification and western blotting was done as described before by us in some detail⁴⁸. Briefly, HEK 293 cells were transiently transfected with wild-type HSP20 by using Polyfect Transfection Reagent (Qiagen). Transfected HEK 293 cells were treated with 10 μM bs906 or 10 μM control peptide for 0, 15, 30, 60 and 120 min. Control and treated transfected HEK 293 cells were then washed thrice with ice cold PBS (phosphate buffered saline) before cellular lysates were prepared. Cellular lysates were prepared in lysis buffer containing 25 mM Hepes, pH 7.5, 2.5 mM EDTA, 50 mM NaCl, 50 mM NaF, μM sodium pyrophosphate, 10% (v/v) glycerol, 1% (v/v) Triton X-100 and Complete™ EDTA-free protease inhibitor cocktail tablets (Roche). Protein concentration of lysates were determined using the Bradford assay and all samples were equalised for protein concentration. Proteins were separated by SOS/PAGE (4-12% Bis-Tris gels) and transferred onto nitrocellulose membranes for Western blotting. Antibodies were used to detect phospho-proteins and native proteins in lysates from control and treated cells

### Phosphodiesterase Assays

PDE activity was measured using a radioactive cAMP hydrolysis assay that has been described previously⁵⁶. [8-³H]adenosine cyclic-3,5′-mono-phosphate was sourced from Amersham Biosciences (Little Chalfont, U.K.) and cyclic-3′,5′-mono-phosphate from Sigma.

### Pulldown Assays.

GST-PDE4 fusions proteins (500 μl of 2 μM) were mixed with 100 μl slurry of 50% (v/v) PBS-washed Glutathione beads for 2 h at 4° C. The beads were pelleted by centrifugation (14,000 g, 1 min) and washed twice with PBS containing 1% Triton X-100 before the addition of equal molar amounts of HSP200-His fusion protein in a 1 mL solution of PBS and Triton X-100 containing 5 mM dithiothreitol. After a 2 h incubation at 4° C. the beads were collected and washed three times with PBS and eluted in 100 μl of 2× Laemmli buffer. Eluates were resolved by SDS-PAGE, and the HSP20-His fusion proteins were detected by immunoblotting.

### Determination of Cellular Size of Cardiac Myocytes

### Manual Measurements:

Cardiomyocytes were plated out on gelatin (Sigma) pre-coated 6-well plates and placed in an incubator at 37° C. and an atmosphere of 5% CO₂. After 24 h the cardiomyocytes were cultured in a serum-free condition for 48 h before experiments. The cardiomyocytes were then challenged for 24 h with either 10 μM isoprenaline alone or isoprenaline following a 30 min pre-treatment with 10 μM rolipram, 10 μM bs906 or 10 μM control peptide. Thereafter, cell size was determined on phase-contrast microscope. About 75 cells from randomly selected microscope images (400×) were captured digitally and analysed. Cross-sectional area was determined by the following formula: cross-sectional area=(radius)²×π. Values are expressed as means±SE of five separate culture preparations. A f-test was performed to compare the two groups, and the P values are indicated.

### ExCELLigence Measurements:

The E-Plate 96 (Roche) was used for these experiments⁴⁵. This comprises a 96-well plate with integral sensor electrode arrays to measure cell impedance. The xCELLigence system was used according to the instructions of the supplier (Roche Applied Science). The core of the xCELLigence system is the E-plate 96: this is a single use, disposable device used for performing cell-based assays on the RTCA SP instrument. E-plate 96 is a 96-well microtiter plate with incorporated gold cell sensor arrays in the bottom, which allows impedance inside each well to be monitored and assayed in real time. The changes in impedance correlate with cell shape changes (short term) or growth (long term) of the cells in each well^{44, 46, 57}. The E-plate 96 has a low evaporation lid design. The diameter of each well is 5.0 mm±0.05 mm, with a total volume of 243±5 μl, approximately 80% of the bottom areas of each well is covered by the circle-on-line-electrodes, which is designed to be used in an environment of +15 to +40° C., relative humidity 98% maximum without condensation⁴⁵. The voltage applied to the electrodes during RTCA measurement is about 20 mV (RMS). The impedance measured between electrodes in an individual well depends on electrode geometry, ion concentration in the well and whether or not cells are attached to the electrodes⁴⁵. In the absence of cells, electrode impedance is mainly determined by the ion environment both at the electrode/solution interface and in the bulk solution. In the presence of cells, cells attached to the electrode sensor surfaces will act as insulators and thereby alter the local ion environment at the electrode/solution interface, leading to an increase in impedance⁵⁷. Thus, the more cells that are growing on the electrodes, the larger the value of electrode impedance. Small changes in impedance can also be recorded when cells temporally change shape or size in response to agonists^{46,57}. The RTCA associated software allows users to obtain parameters such as: average value, maximum and minimum values, standard deviation (SD), half maximum effect of concentration (EC50), half maximum inhibition of concentration (IC50) and cell index (CI)⁵⁷. Cardiomyocytes were counted using a hemocytometer and adjusted to the desired concentration. An initial population of 40000 cardiomyocytes/well was plated out on the laminin (BD Biosciences) pre-coated 96-well e-plate in triplicates after background measurements were taken, Briefly after 48 h of culture in the serum-free medium, cardiomyocytes were treated with either 10 μM isoprenaline alone or isoprenaline following a 30 min pre-treatment with 10 μM rolipram, 10 μM bs906 or 10 μM control peptide. All chemicals were dissolved in DMSO (final concentration 1:1000), including control well, 0.001% of DMSO was added into each well. The cultures were continuously monitored for up to 48 h and the impedance as reflected by cell index (CI) values were automatically recorded every 30 minutes using the real-time cell-electronic sensing (RT-CES) system (Roche Applied Sciences). The results were expressed by normalised CI which are derived from the ratio of CIs before and after the addition of compounds.

### Hsp20/PDE4D5 ELISA Assay

His-HSP20 and GST-PDE4D5 were purified as previously described. GST-PDE4D5 was diluted to a final concentration of 4 μg/ml in coating buffer (PBS with 1 mM DTT, 0.6 mM PMSF, Roche protease inhibitor cocktail) and 50 μl was added to half of the wells of a 96-well low-flange white flat bottom polystyrene high-bind microplate (Corning). Control wells containing just coating buffer alone were also included. Plates were incubated over-night at 4° C. Protein solution was removed from the plate and 50 μl of blocking buffer (PBS with 0.03% milk powder, 1 mM DTT, 0.6 mM PMSF, 0.05% Tween20) was added to each well. Plates were incubated at room temperature for 1 hour. Purified His-HSP20 was diluted to a concentration of 0.5 μg/mL in blocking buffer. Disrupter peptide and control peptide were added to aliquots of this protein solution at increasing dilutions and samples were placed on a rotator and incubated at 4° C. for 1 hour. Plates were washed three times with PBST (PBS with 0.05% Tween20) and 50 μl His-HSP20/peptide preparations were added to wells and plates were incubated for 2 hours at 4° C. Plates were then washed three times with PBST before anti-HSP20 antibody (Upstate 07-490), diluted 1:2000 in blocking buffer, was added, and plates were incubated for 1 hour at room temperature. After washing three times with PBST, anti-Rabbit HRP conjugated secondary antibody (Sigma), diluted 1:3000 in blocking buffer was added and plates were incubated for 30 mins at room temperature. Plates were washed three times with PBST and 50 μl of ECL Western blotting reagent (Pierce) was added. Plates were read as soon as possible using the luminescence program on a Packard Fusion plate reader (1 s reading per well). Control (His-HSP20 alone) values were subtracted from experimental values and the results plotted using Excel (Microsoft).

### HSP20-FRET

Primary cultures of ventricular cardiac myocytes were prepared from 1-3 day old Sprague-Dawley rats, as described above. Cells were seeded onto sterile glass coverslips precoated with 1 μg/cm² mouse laminin (BD Biosciences) at a density of 700,000 cells per well, and transfected using Transfectin Lipid Reagent (BioRad), according to manufacturer instructions. Cells were transfected on the 3^{rd} day in culture, and FRET imaging was performed 24-48 hours following transfection. Measurements were obtained from cells buffered in a solution of 125 mM NaCl, 5 mM KCl, 20 mM HEPES, 1 mM Na₃PO₄, 1 mM MgSO4, 1 mM CaCl₂ and 5.5 mM glucose, pH 7.4, at room temperature. Cells were stimulated in real time with 1 nM isoproterenol or 10 μM rolipram, and imaged on an Olympus IX71 inverted microscope with a 1000× oil immersion objective (Zeiss). The microscope was equipped with a beam splitter optical device (Photometrics). Images were acquired using MetaFluor software (Molecular Devices). FRET changes were measured following background subtraction by dividing the intensity of emissions at 480 nm and 545 nm, following excitation at 440 nm. Data are expressed as dR/R₀, where R=fret ratio at time t (sec), R₀=fret ratio at time zero, and dR=R−R₀. Graphs are presented as means+/−SEM.

### SPOT Synthesis of Peptides and Overlay Experiments.

This was done as described in detail elsewhere⁴¹.

### Antibodies

The following antibodies were used in western blotting analysis: anti-HSP20-Upstate (07-490), anti-phospho-S16 HSP20-Abcam (ab58522), anti-6×His-tag HRP-Abcam (ab1187), alpha tubulin HRP-Abcam (ab40742). Secondary antibodies used: anti-mouse HRP-GE Healthcare (NXA931), anti-rabbit-Sigma-Aldrich (A6154).

### His-Hsp20 Purification

The full length Hsp20 sequence was cloned into a pET28c vector (Novagen) and transformed into competent BL21 cells (Invitrogen, Paisley). Cells were grown until OD600=1 prior to 1 mM of IPTG being added to induce expression. Cells were grown for a further 24 hours at 25° C. and the resultant protein was purified using nickel affinity chromatography. The protein product was analysed through SDS-PAGE and western blotting techniques,

### SDS-PAGE & Western Blotting

SDS-PAGE analysis was done on NuPage® pre-cast gels X-cell apparatus (Invitrogen, Paisley) using MES-SDS running buffer, Samples were reduced in Laemmli 2× loading buffer (4% SOS) with 5% β-mercaptoethanol. For Western Blotting (WB) analysis, samples were transferred using NuPage® X-cell blotting module onto a nitrocellulose membrane. Membranes were blocked using 5% Milk in 1×TBST (w/v). Antibodies were incubated in 1% milk in 1×TBST (w/v) for either 1 hour at room temperature or overnight at 4° C. Signals were detected using enhanced chemiluminescence system (Pierce) and developed on an X-omat film developer. Subsequent densitometry analysis was carried out using Quantity One (Bio-Rad).

### Cell Culture

SH-SY5Y cells (ATCC-CRL-2266) were grown in Dulbecco's Modified Eagle's Medium (DMEM) and F12-Ham's at a 1:1 ratio, media was supplemented with 10% (v/v) fetal bovine serum (FBS), 1% (v/v) L-Glutamine, 1% (v/v) Minimum Essential Medium—With Non-essential Amino Acids (MEM-NAA) and 1% (v/v) Pen/strep. Cells were cultured in a humidified, 5% (v/v) CO2, 37° C. incubator.

### Disrupter Nucleofection

SH-SY5Y cells were nucleofected using Cell Line Nucleofector® Kit-V in accordance with manufacturers protocol (Amaxa Biosystems). Setting A-23 on the Nucleofector I device was used which increases cell viability. For each assay, 500 μM of disrupter peptide was incubated with 100 μl of nucleofection solution containing approximately 2×10⁶ cells. After nucleofection cells were re-suspended in 500 μl of culture media before being transferred to a 6 well plate with a final volume of 2 ml per well. Cells were incubated for 2 hours prior to harvesting with 3T3 containing protease and phosphatase inhibitors (Roche).

### Results

### Example 1

### PDE4 Activity Modulates PKA Mediated phospho-HSP20 Levels in Response to Isoprenaline Treatment

We performed our initial experiments in a model HEK cell line that stably overexpresses β₂-adrenoceptors (HEK-B2 cells). This is well-characterised as regards its PDE4 isoform complement and has proved effective in uncovering the role of a PDE4D5-βarrestin complex in regulating β₂-adrenoceptor functioning in cardiac myocytes³².

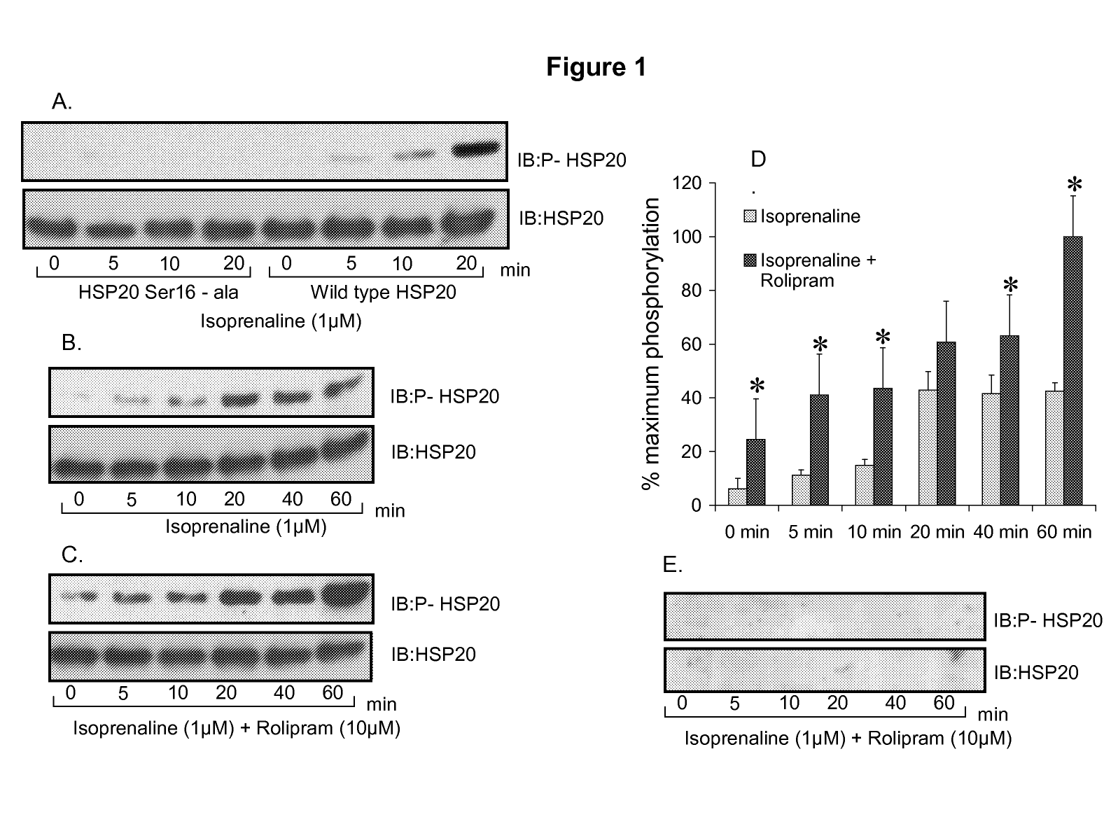

We transiently transfected HEK-82 cells with wild type (wt) HSP20 wt and treated them with isoprenaline (1 μM) for indicated times, which led to the time dependent phosphorylation of Hsp20 at Ser16 as detected using a specific phospho-antibody (FIG. 1A). In contrast to this, when we transfected these cells with a phospho-null mutant form, Ser16Ala-Hsp20, then no such change was observed (FIG. 1A). In order to evaluate whether PDE4 activity played a role in regulating this phosphorylation process in Hsp20-transfected HEKB2 cells, cells that had either been pre-treated or not with the PDE4 selective inhibitor rolipram were subsequently challenged with isoprenaline (1 μM) (FIGS. 1B and C). Doing this, we see that selective PDE4 inhibition increased isoprenaline-stimulated HSP20 phosphorylation (FIG. 1D). Indeed, we noted that PDE4 inhibition alone, effected by rolipram treatment, was sufficient to produce a clear increase in phospho-HSP20 levels when compared to that seen in resting cells (FIG. 1D). Endogenous levels of phospho-HSP20 or HSP20 were not detectable in HEKB2 cells (FIG. 1E).

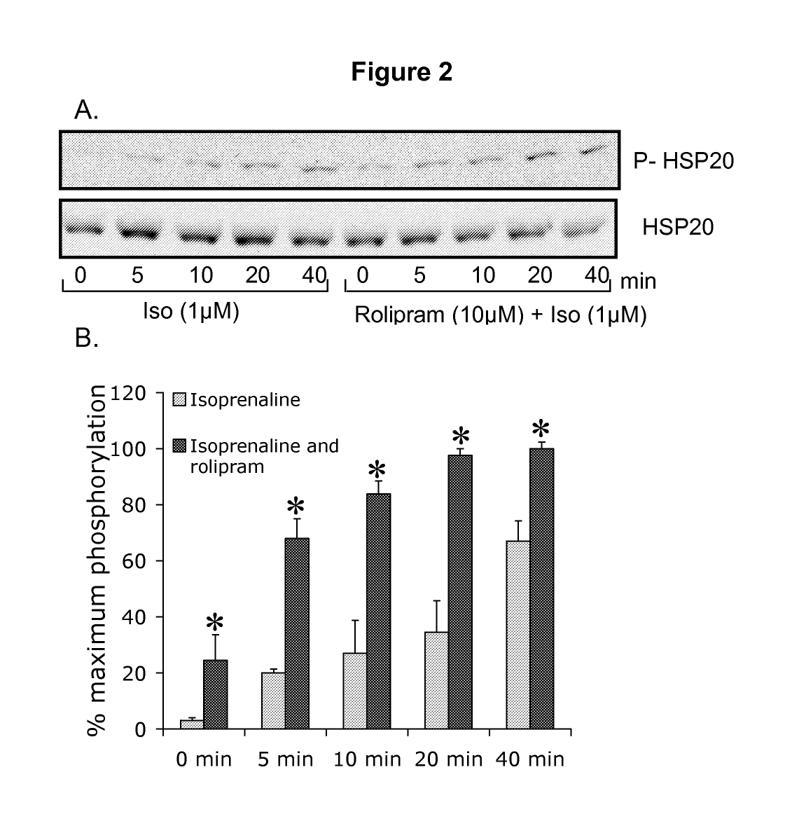

To determine whether inhibition of PDE4 activity had similar effects on endogenous phospho-HSP20 levels in heart cells, similar experiments were undertaken using neonatal cardiomyocytes (FIG. 2). Similarly, isoprenaline-challenge of neonate myocytes induced an increase in endogenous phospho-HSP20 levels (FIG. 2A) and this effect was markedly augmented with rolipram pre-treatment (FIG. 28). Furthermore, in cardiomyocytes, PDE4 inhibition alone was sufficient to produce a marked increase in phospho-HSP20 levels (FIGS. 2A and B).

To determine whether phosphorylation of HSP20 at Ser16 was PKA mediated, both HEKB2s and cardiomyocyes were pre-treated with PKA inhibitors (data not shown) prior to stimulation with either isoproterenol alone or isoproterenol with rolipram pre-treatment. Inhibition of PKA activity by either of H89 or KT5720 treatment markedly attenuated HSP20 phosphorylation, consistent with Ser 16 phosphorylation of HSP20 being PKA mediated in both these cells.

### Example 2

### PDE4 Isoforms Associate Directly with HSP20

The ability of PDE4 isoforms to shape cAMP gradients within cells depends on their unique ability to exhibit selective anchoring in specific microdomains via protein-protein interactions^{24, 27}. As rolipram had such a profound effect on the PKA phosphorylation of HSP20, we set out to determine if PDE4 formed a complex with HSP20 either directly or via another protein scaffold.

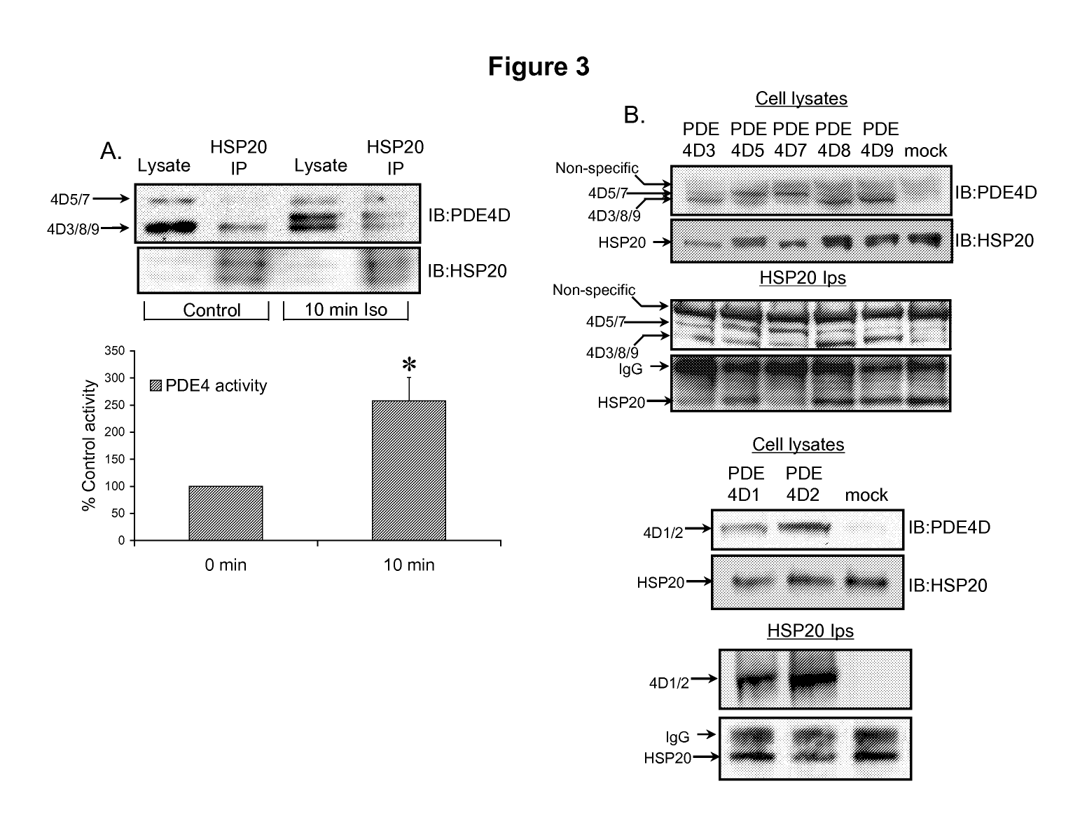

Immunohistochemical studies indicated that HSP20 and PDE4D exist in the same cellular compartment in neonatal cardiac myocytes as both proteins localised with □-actinin (data not shown). Immunopurification (Ips) of HSP20 from cardiac myocytes were screened for associating PDE4 by Western blotting analysis and cAMP PDE assay (FIG. 3A). HSP20 immunoprecipitates from resting and isoprenaline treated cells were shown to contain PDE4D isoforms (FIG. 3B: upper panels). When the HSP20 immunoprecipitates were screened for PDE4 activity (FIG. 3A; bar chart), samples isolated from isoprenaline treated cells exhibited a 2.5 fold increase in the associated PDE4 activity when compared to Ips from untreated lysates. This could be as a result of activation of the HSP20-associated long PDE4 isoforms by established means through PKA phosphorylation³³ or by an increase in HSP20-PDE4 association per se, which has been shown to alter interactions between specific PDE4 isoforms and both Nudel³⁴ and mAKAP³⁵.

In an attempt to discover which PDE4D isoforms might potentially associate with HSP20 we co-transfected HEKB2 cells with HSP20 and seven different PDE4D isoforms (4D1, 4D2, 4D3, 4D5, 4D7, 4D8 and 4D9). As before, we immunopurified HSP20 and blotted for the presence of transfected isoforms (FIG. 38). To our surprise, all of the transfected PDE4D isoforms co-purified with HSP20 suggesting a common association site within the conserved parts of this enzyme family. Indeed, we noted that all of the endogenously expressed PDE4D isoforms from HEK-B2 cells were detected in Hsp20 immunopurified from these cells (FIG. 3B).

To evaluate further Hsp20 association with PDE4, we set out to determine whether they interacted directly. In order to do this we expressed Hsp20 and various PDE4 species in E. coli and purified them for use in pull-down assays. We show that recombinant His-tagged HSP20 co-purifies with GST-PDE4B3, GST-PDE4D3 and GST-PDE4D5 but not GST alone (data not shown). Additionally, we performed the converse experiment and first did pull-downs of HSP20, using its His tag, and then tested for co-purification of PDE4 species. Doing this, we saw co-immunopurification of Hsp20 with MBP-PDE4A5, MBP-PDE4A4 and MBP-PDE4D3 but not with the MBP tag alone.

These data indicate that HSP20 interacts directly with PDE4 and this association is likely a ‘pan-family’ one, as we can show direct interactions here with members of the PDE4A/B/D sub-families. This suggests that Hsp20 binds directly to a region that is conserved in all PDE4s.

### Example 3

### The HSP20 Binding Site Lies within the Conserved Catalytic Region of PDE4s

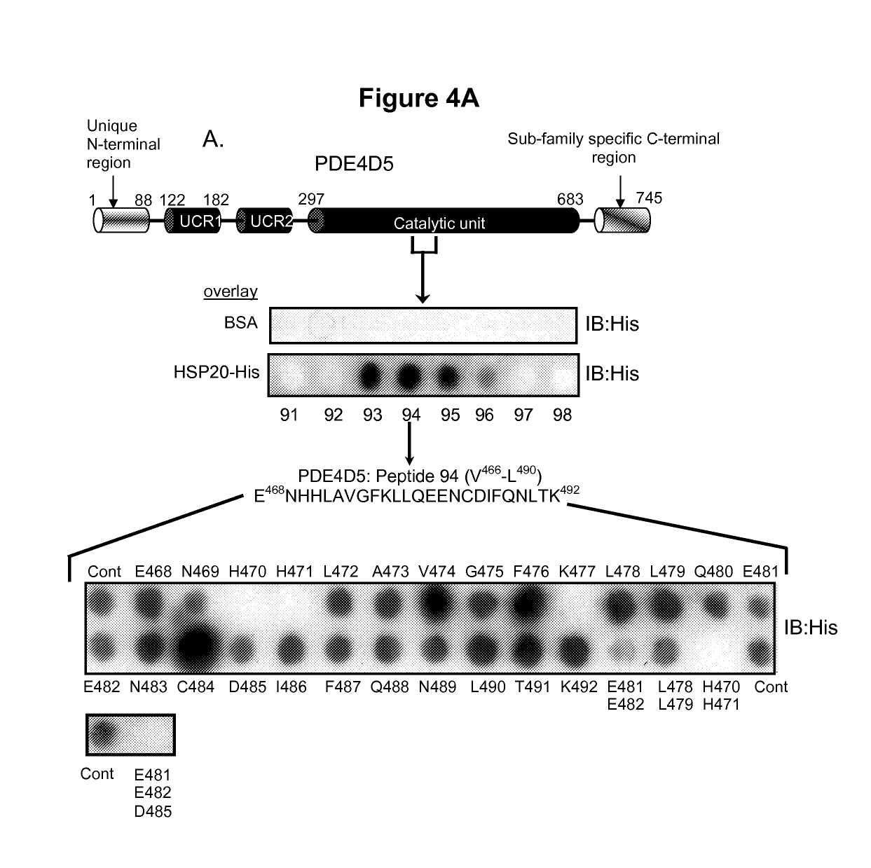

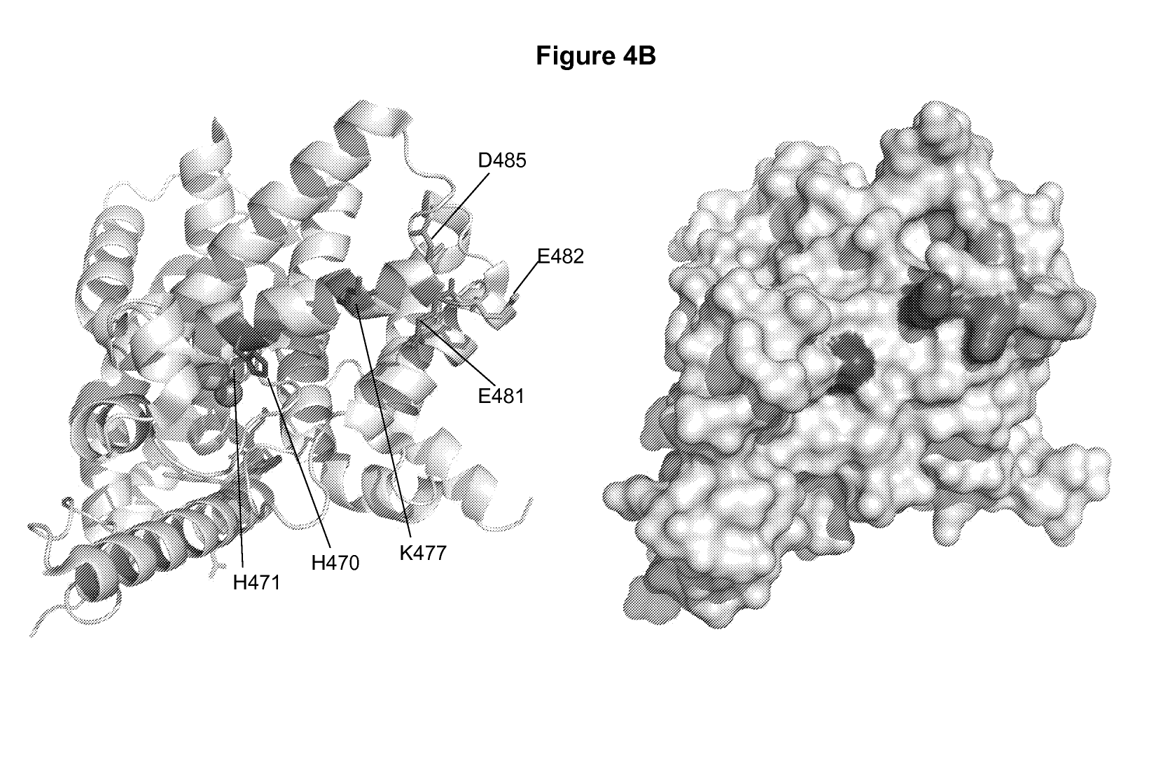

PDE4 isoform localisation invariably depends on protein-protein interactions, with PDE4s shown to associate with a diverse group of interacting partners such as RACK1^{32, 36}, βarrestin³⁶, β₁-Ar³⁷, DISC1³⁸, the p75 neurotrophin receptor³⁹ and the cardiac IKs potassium channel⁴⁰. One method that has been pivotal in mapping the sites of association between PDE4s and their binding partners is peptide array³⁶. SPOT immobilised peptide libraries of PDE4 sequences have been utilised to accurately predict binding domains for partner proteins. Here we have employed this novel technique to define the HSP20 binding site within the sequence of PDE4D5, an isoform that is endogenously expressed in cardiomyocytes. Peptide arrays of overlapping 25-mer peptides, sequentially shifted by 5 amino acids and spanning the entire sequence of PDE4D5, were incubated with purified recombinant HSP20-6his. Dark spots represent positive areas of HSP20 interaction whereas clear spots are negative for the association (FIG. 4A). While no signal was observed when the arrays were overlaid with BSA, positive signals were obtained for 25mer peptides 93 to 96 following HSP20-6His overlay. Peptides 93 to 96 span the amino acid sequence Y⁴⁶¹-K⁵⁰⁰ within the conserved catalytic domain of PDE4D5 (FIG. 4A: middle panels). Gratifyingly, this result is consistent with the data from FIG. 3 and Supplemental FIG. 3, in that this region is identical in all isoforms from the four sub-families of PDE4s.

To gain insight into which amino acids within PDE4D5 might be critical in binding to HSP20, we focussed on the E⁴⁶⁸-K⁴⁹² region of PDE4D5 and using a ‘parent’ 25-mer peptides generated 25 progeny of this peptide where successive ones contained a sequential single substitution of alanine to provide an alanine-scanning array. This has been used successfully before by us to define the binding sites on PDE4D5 for both RACK1 and βarrestin, for example^{36, 41}. Such a library of peptides was once again probed with HSP20-6His (FIG. 4A: lower panel). This identified three regions likely to be important for HSP20 association with PDE4D5, namely the double histidine (H⁴⁷⁰, H⁴⁷¹), K⁴⁷⁷ and the acidic region consisting of E⁴⁸¹, E⁴⁸² and D⁴⁸⁵. Superimposing these residues on the available crystal structure of the PDE4 catalytic domain shows that, save H471, all of these amino acids are surface associated and potentially could thus form a surface available for HSP20 docking (FIG. 4B).

### Example 4

### PDE4 Activity Controls cAMP Dynamics in the Vicinity of HSP20

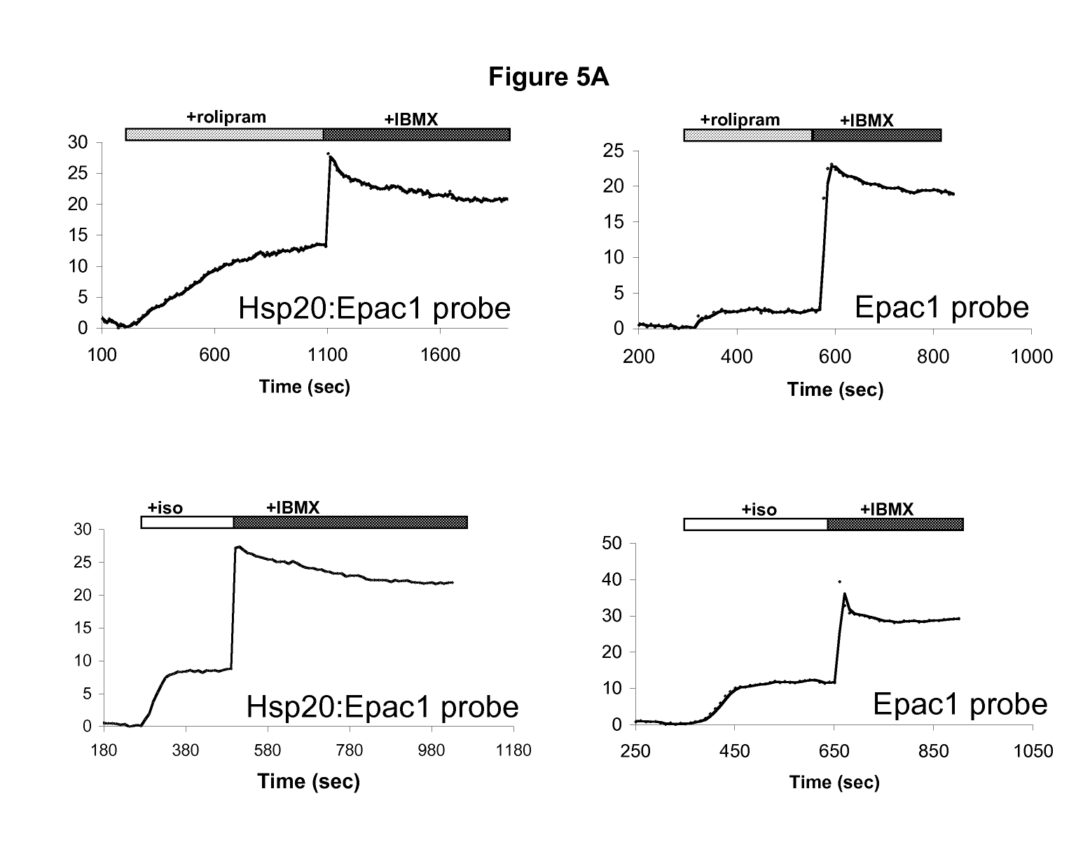

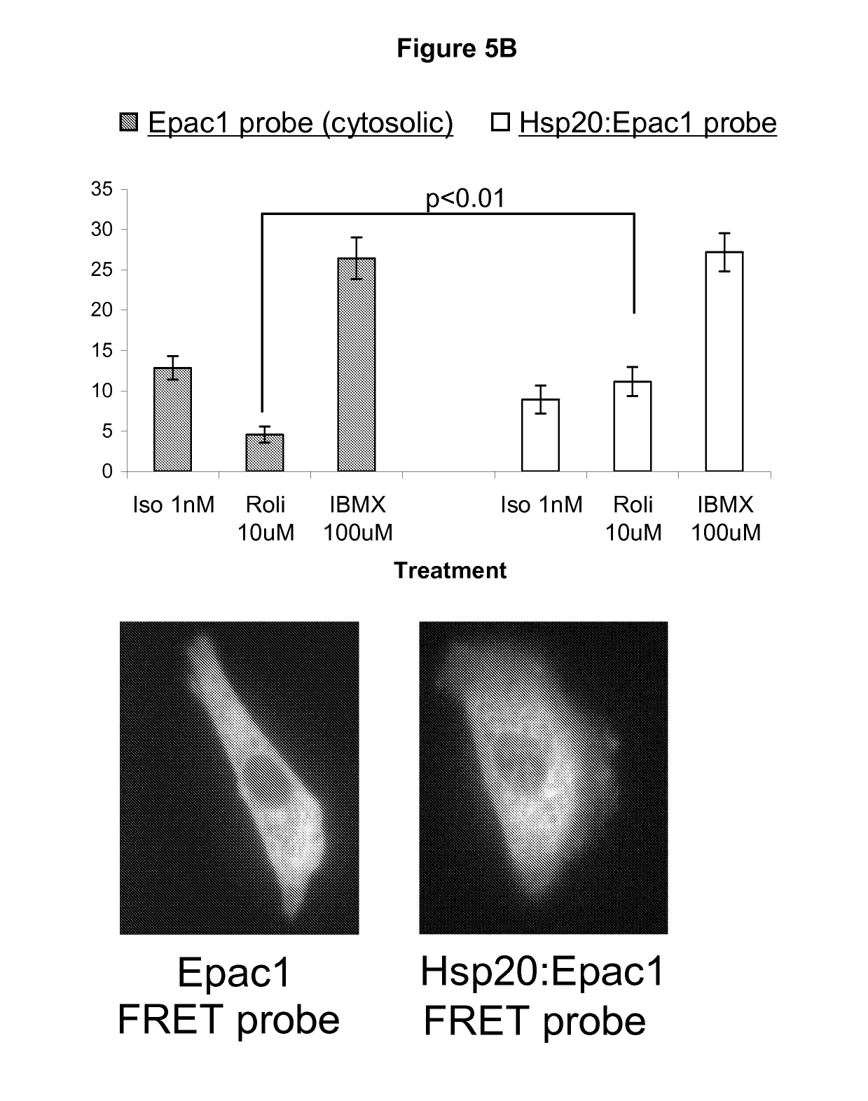

Genetically encoded cAMP reporters have been extensively utilised to monitor the effects of PDE inhibition on cAMP dynamics following receptor stimulation²⁴. One such reporter is based around the cAMP binding domain of the exchange factor activated by cAMP (EPAC1), and has been used as a highly sensitive cAMP indicator in cultured cells⁴². In order to compare global cAMP changes with those uniquely associated with HSP20, we constructed a fusion between HSP20 and the Epac-based FRET probe and compared cAMP dynamics of this targeted construct with the original cytosolic reporter (FIG. 5). Following treatment with the non-specific PDE inhibitor IBMX, the HSP20 fusion reporter had an identical maximal response when compared with to the cytosolic probe, suggesting that both constructs were equally efficient in measuring cAMP concentration (FIG. 5A).

Isoprenaline treatment produced expected increases in cAMP concentration that were of similar magnitude for both probes (FIG. 5B). However, the increases in cAMP produced following treatment with rolipram were clearly greater in the HSP20 targeted reporter (FIG. 5B). As both probes were expressed at the same level (FIG. 5B, lower panel), these results suggest that PDE4 enzymes dominate in modulating cAMP flux in the cellular compartment occupied by HSP20. One extrapolation of this observation is that PDE4 should also regulate PKA activity localised with HSP20 and this supports the data presented in FIGS. 1C and 2, where rolipram treatment alone is sufficient to trigger the PKA mediated phosphorylation of HSP20 in a cellular context.

### Example 5

### Peptide Disruption of the HSP20-PDE4D Complex

Peptide array technology has allowed us to identify the putative docking site for HSP20 on the catalytic unit of PDE4D5 (FIG. 4A) in a region spanning V⁴⁶⁶-L⁴⁹⁰. Subsequent alanine scanning analysis highlighted several key residues within this sequence (H⁴⁷⁰, H⁴⁷¹, K⁴⁷⁷, E⁴⁸¹, E⁴⁸² and D⁴⁸⁵) as of being potentially important for HSP20 association. Previous work from our laboratory and those of others has shown that cell-permeable analogues of 25mer peptides identified in this manner, can be used to disrupt signalling complexes within cells to effect specific functional outputs such as phosphorylation of the β-adrenergic receptor by PKA³² and the phosphorylation of βarrestin by ERK MAPK⁴³.

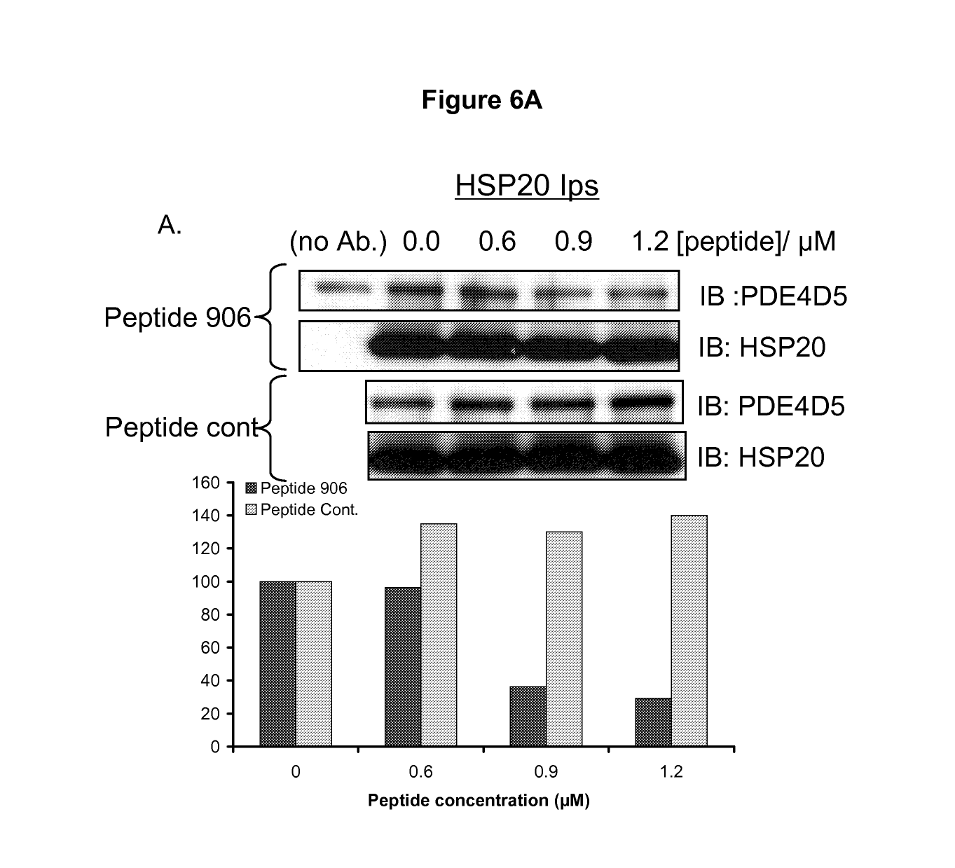

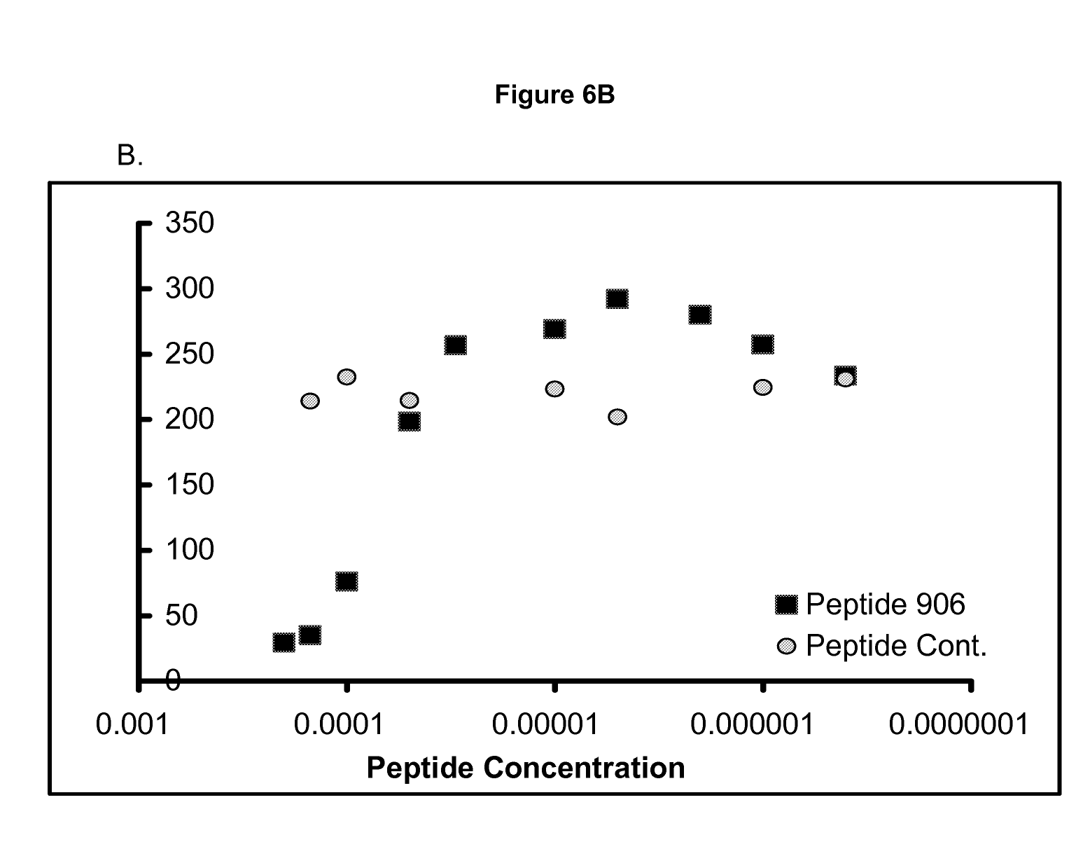

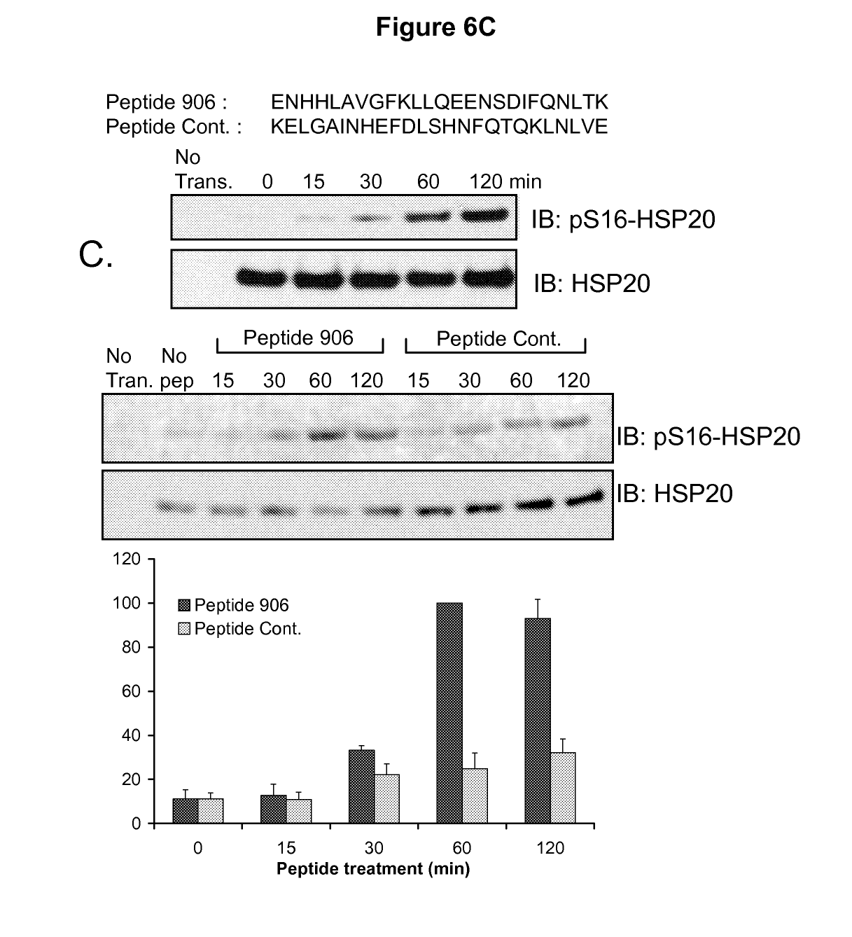

In this study, we utilised the HSP20 docking sequence from PDE4D5 encompassing residues E⁴⁶⁸-K⁴⁹² to manufacture a stearoylated, cell-permeable disrupter peptide (peptide 906). We also synthesised a scrambled version of stearoylated, peptide 906 that had the same net weight and charge (Peptide Cont). In these reagents both peptides had a C-terminal stearate group that has been shown to allow transport across the cell membrane⁴³. ‘Peptide 906’ but not ‘peptide Cont’ attenuated the association between PDE4D5 and HSP20 when measured in immunoprecipitation experiments from cellular lysate (FIG. 6A) and in ELISA experiments utilising purified proteins (FIG. 6B). Gratifyingly, treatment of cells with ‘peptide 906’ but not ‘peptide Cont’ caused a robust and significant increase in the phosphorylation of HSP20 at Ser16 (FIG. 6C), suggesting that disruption of the PDE4 component from the HSP20 complex released the tonic inhibition of PKA by preventing PDE4 sequestration. These data are consistent with our studies showing that treatment with rolipram alone sufficed to increase the phosphorylation of Hsp20 at Ser16 (FIG. 1). Thus we can infer that basal concentrations of cAMP are sufficient to trigger HSP20 phosphorylation by PKA in the absence of localised PDE4 activity, pointing to sequestered PDE4 as being a crucial regulator of Hsp2 phosphorylation by PKA.

### Example 6

### Peptide Disruption of the HSP20-PDE4 Complex Protects Against Hypertrophy in Cultured Cardiac Myocytes

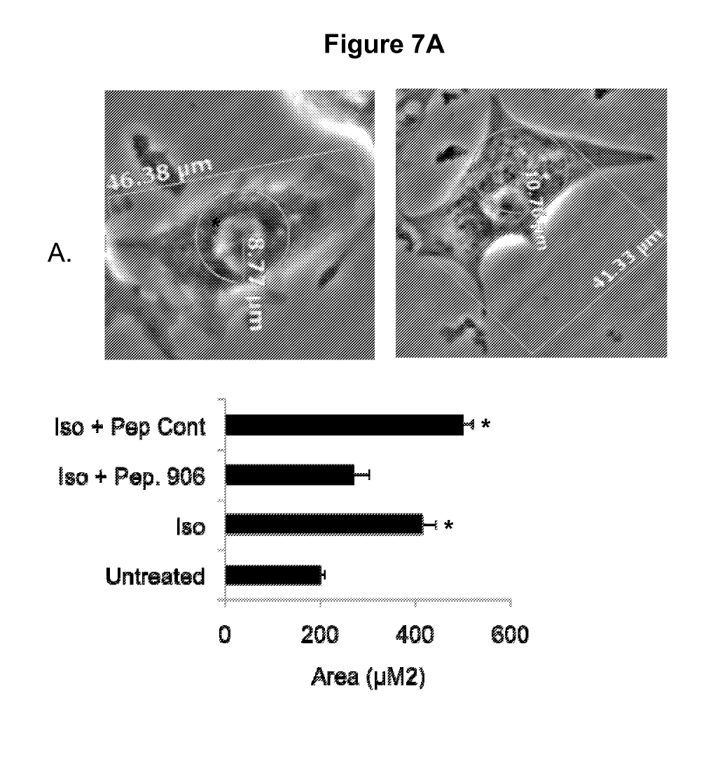

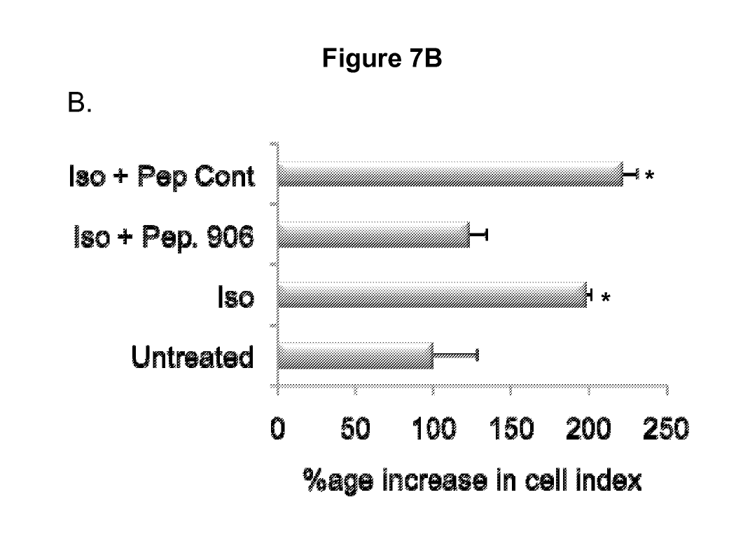

Chronic stimulation of β-adrenergic receptors is known to promote cardiac remodelling, which results in altered cellular signalling leading to hypertrophy¹⁰. As we can induce the phosphorylation of HSP20 by treating cells with ‘peptide 906’ to disrupt the HSP20-PDE4 complex (FIG. 6C), we were interested to see if the peptide might also be able to attenuate the hypertrophic response induced by chronic β-agonist stimulation of neonatal cardiac myocytes. We utilised two assay methods to measure the effectiveness of ‘Peptide 906’ to confer protection against β-adrenergic receptor-triggered hypertrophy. Firstly, we employed a traditional approach where we physically measured the cross-sectional areas of the cardiac cells (FIG. 7A) and, secondly, a novel approach employing exCELLigence technology^{44, 45}, which measures impedance on the surface of cell culture plates (FIG. 7B). The latter technique provides a dynamic, non-invasive, label-free analysis of the relative size of cells (surface area on plate) providing that they are terminally differentiated. Briefly, the larger the cells become the more the resistance they impart on the cell plate and this can be measured and used as an index of cell size⁴⁶. Both techniques produced strikingly similar data, showing that a 24 hour treatment of cardiac myocytes with isoprenaline resulted in a marked increase in cell size that could be rescued by the HSP20-PDE4 complex disrupter ‘peptide 906’ but not the control, scrambled peptide (FIGS. 7 A+B). As ‘peptide 906’ (FIG. 6C) induces phosphorylation of HSP20 by PKA and this is recognised as the protective step in the ability of HSP20 to confer cardioprotection¹⁷, it appears that the association between HSP20 and PDE4 acts as a brake on this process. Thus disruption of this complex may represent a novel therapeutic route for the prevention of hypertrophy.

### Example 7

### Peptide Array to Identify Shorter Disrupter Peptides

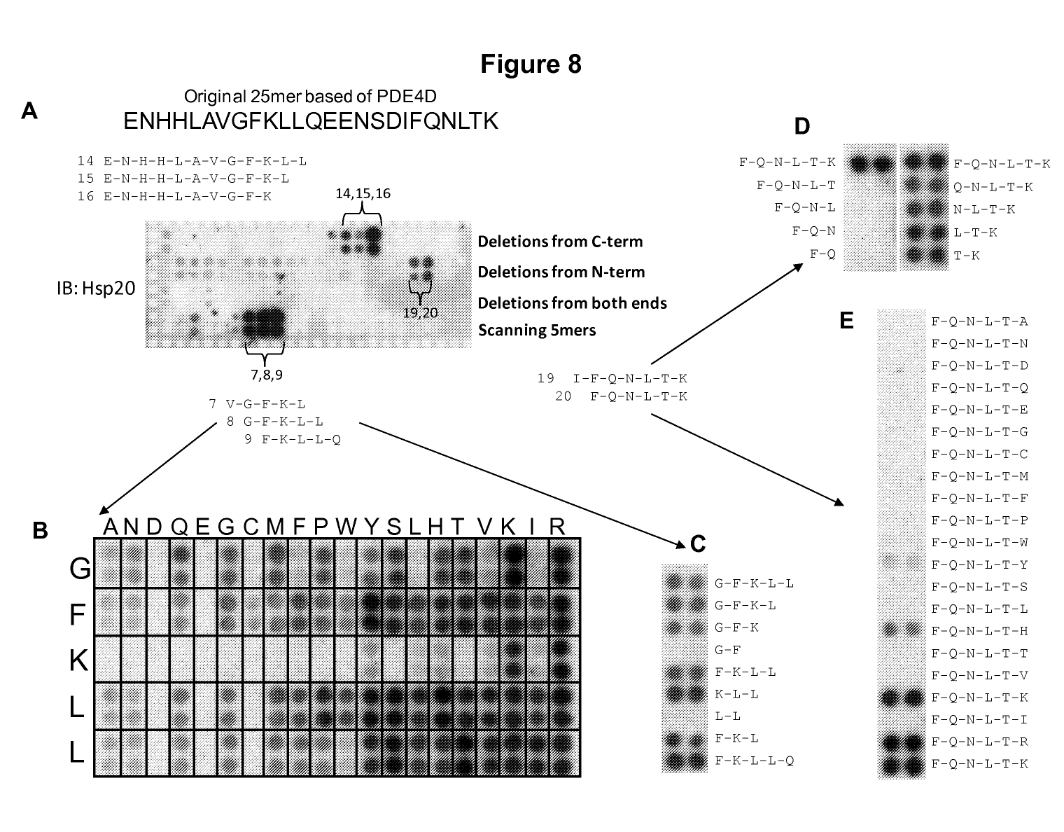

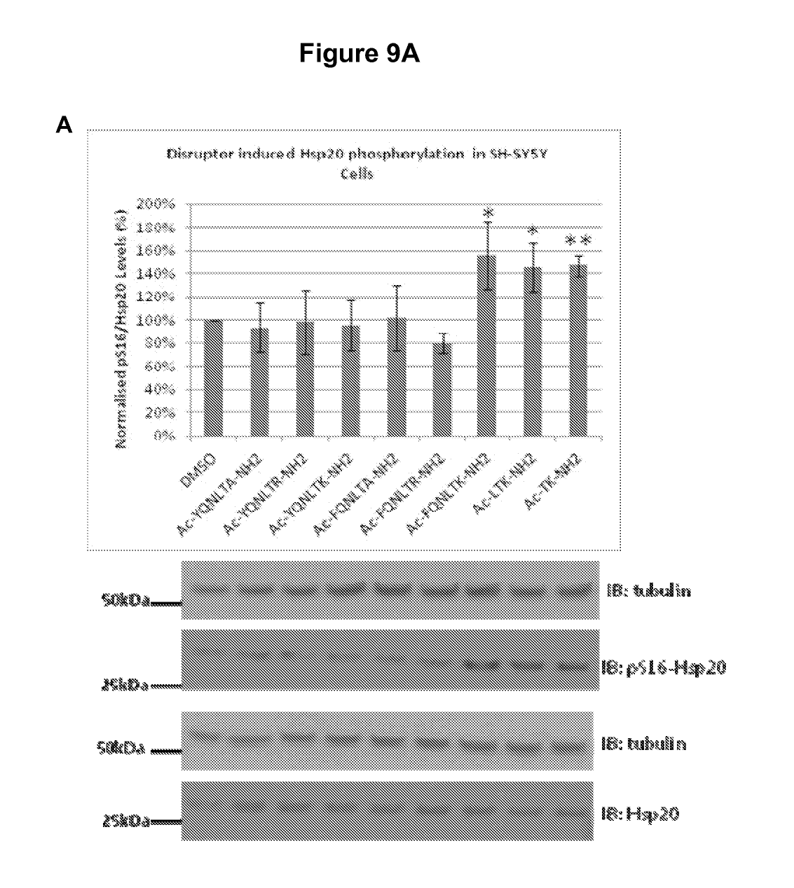

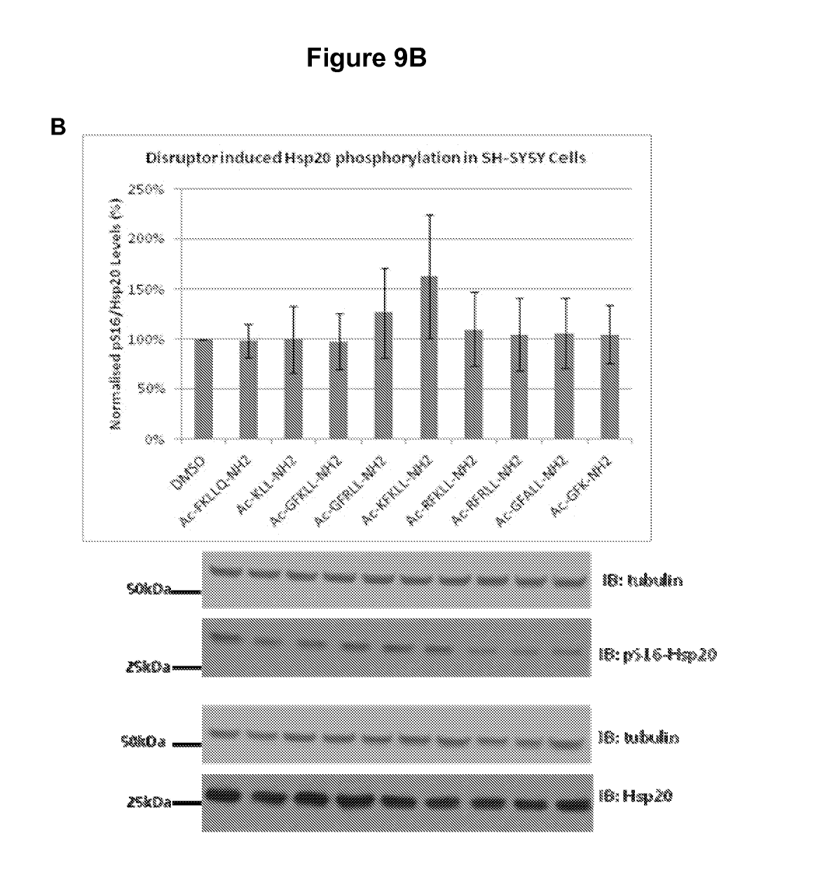

The particular peptide domains responsible for binding of Hsp20 was further characterized using peptide array technology, in order to find the smallest possible size that still binds Hsp20, Truncations of the 25mer sequence E⁴⁶⁸-K⁴⁹² region of PDE4D5 were carried out from both C and N-term directions. Scanning of the 25mer sequence utilised methodologies analogous with ‘peptide walking’, where we took the original 25mer sequence and broke this down into 5mers which ran sequentially form an N—C terminal direction in increments of 1 amino acid at a time. Once positively interacting domains were established subsequent rounds of substitutions were carried out to determine which residues were essential for mediating binding of Hsp20. As shown in FIG. 8A, peptides containing residues F⁴⁸⁷-K⁴⁹² and V⁴⁷⁴-Q⁴⁸⁰ interacted particularly strongly with HSP20.

The substitution of native residues yielded expected results in that charged residues such as the lysine from GFKLL (SEQ ID NO: 42) (see e.g. FIG. 8C) and FQNLTK (SEQ ID NO: 4) (see e.g. FIG. 8E) were essential in mediating binding and they only retained an ability to bind Hsp20 when the similarly charged residues arginine or histidine were introduced.

Subsequent rounds of truncation of both GFKLL (SEQ ID NO: 42) (FIG. 8C) and FQNLTK (SEQ ID NO: 4) (FIG. 80) demonstrated the minimum peptide size required to induce binding of Hsp20. For GFKLL (SEQ ID NO: 42) the GFK and KLL portions of the 5mer were the shortest size whilst the removal of the lysine residue prevented binding of Hsp20 as demonstrated through the inability of Hsp20 to bind GF or LL.

The FQNLTK (SEQ ID NO: 4) peptide could also be truncated down to shorter peptides in an N to C terminal direction with the di-peptide TK still retaining the ability to bind Hsp20 (FIG. 8D). Truncations in the C to N terminal direction did not retain any Hsp20 binding capacity, further demonstrating the importance of the lysine residue in mediating the interaction (FIG. 8D).

### Example 8

### Cell Based Hsp20-PDE4D Disruptor Assay

From the peptide array data a list of peptides was compiled and synthesised by Thinkpeptides.com to be used in cell based assays, as shown in Table 1 below.

TABLE 1
Candidate Hsp20-PDE4 Disruptor Peptides. A list of candidate peptides
collated from the peptide array data were synthesised by Thinkpeptides.com to allow
us to test for any disruptor activity. Peptides are synthesised with an acetyl group at
the N-terminal and the amine group at the C-terminai in order to resemble SPOT
synthesised peptides. Several control peptides which showed no interaction with
Hsp20 were synthesised to serve as negative controls. A protein BLAST of the non-
native substituted peptides was also carried to check for sequence conservation and
to check the possibility for off-target interactions. The native PDE4D sequences
FQNLTK (SEQ ID NO: 4) and FKLLQ (SEG ID NO: 43) are highlighted in bold, The
FKLLQ sequence was also shown to be conserved among other PDE4 isoforms.

| | | Peptide Array | |
|---|---|---|---|
| No | Peptide | interaction | BLAST (hits with 100% match) |
| 1 | Ac-YQNLTA.NH2 | untested | YQNLT (SEQ ID NO: 73) 316 hits (Spectrin) |
| | (SEQ ID NO: 72) | | |
| 2 | Ac-YQNLTR-NH2 | untested | YQNLTR (SEQ ID NO: 74)-312 hits (Spectrin) |
| | (SEQ ID No: 74) | | |
| 3 | Ac-YQNLTK-NH2 | + | YONLTK (SEQ ID NO: 75)-304 hits (Glutathione |
| | (SEQ ID NO: 75) | | reductase) |
| 4 | Ac-FQNLTA-NH2 | − | FQNLT (SEQ ID NO. 47)-108 hits (TLR8) |
| | (SEQ ID NO: 49) | | |
| 5 | Ac-FQNLTR-NH2 | + | FQNLTR (SEQ ID NO 67)-115 hits (Tight junction |
| | (SEQ ID NO: 67) | | protein 3, Spectrin) |
| 6 | Ac-FQNLTK-NH2 | + | FQNLTK (SEQ ID NO 4)-120 hits (PDE4D-1-9) |
| | (SEQ ID NO: 4) | | |
| 7 | Ac-LTK-NH2 | + | Sequence too short |
| 8 | Ac-TK-NH2 | + | Sequence too short |
| 9 | Ac-FKLLQ-NH2 | + | FKLL1 (SEQ ID NO 43)-105 hits (IP3R, PDE4A1- |
| | (SEQ ID NO: 43) | | 2-3-5, PDE4C1-2-3, PDE4B1-2-3, cytochrome |
| | | | P450) |
| 10 | Ac-KLL-NH2 | + | Sequence too short |
| 11 | Ac-GFKLL-NH2 | + | GFKLL (SEQ ID NO: 42)-135 hits (microtubule- |
| | (SEQ ID NO: 42) | | actin cross-linking factor 1, fibronectin) |
| 12 | Ac-GFRLL-NH2 | + | GFRLL (SEQ ID NO: 76)-115 hits (SET binding |
| | (SEQ ID NO: 76) | | factor, glutamate receptor GluR-5) |
| 13 | Ac-KFKLL-NH2 | + | KFKLL (SEQ ID NO 77)-100 hits (E3 ligase |
| | (SEQ ID NO: 77) | | HERC2) |
| 14 | Ac-RFKLL-NH2 | + | RFKLL (SEQ ID NO 78)-114 hits (FAT tumor |
| | (SEQ ID NO: 78) | | suppressor, PI4Ka) |
| 15 | Ac-RFRLL-NH2 | untested | RFRLL (SEQ ID NO 79)-145 hits (Nesprin-1. |
| | (SEQ ID NO: 79) | | votage-dependent T type calcium channel) |
| 16 | Ac-GFALL-NH2 | − | GFALL (SEQ ID NO: 80)-115 hits (Reverse |
| | (SEQ ID NO: 80) | | Transcriotase, K+ voltage gated ion channel, |
| | | | FBOX22) |
| 17 | Ac-GFK-NH2 | + | Sequence too short |

To determine the efficacy of these peptides in inducing Hsp20-PDE4D complex disruption and thus increasing Hsp20 phosphorylation, we utilised SH-SY5Y human neuroblastoma cell line which readily express both proteins and are easily detectable through Western Blotting techniques. These neuronal-like cells are used extensively in a variety of in vitro models to study neurological conditions such as Alzheimer's disease and schizophrenia.

As the cell permeability of the peptides is unknown we used an electroporation technique to introduce the peptides into the SH-SY5Y cells. The Amaxa cell-line specific Nucleofector Kit V (Lonza) was used to transfect the peptides into the cells. However as this procedure does not deliver precise quantities of peptide into each cell we used relatively high concentrations of the candidate disrupters in order to exacerbate any effect induced by the peptides. Each assay used a final concentration of 500 μM of peptide in nucleofector solution containing approximately 2×106 SH-SY5Y cells. As peptides were dissolved in 100% DMSO, control transfections with DMSO only were used to compare any changes in phospho-Hsp20 levels. After each transfection the solution was reconstituted in culture media and incubated for 2 hours to allow for cell adhesion prior to the harvesting of viable cells.

The data shows that only 3 of the initial 8 compounds tested induced a significant increase in pS16-Hsp20 levels (FIG. 9A). These three peptides (FQNLTK (SEQ ID NO: 4), LTK and TK) all contain a C-terminal lysine residue, showing the importance of this motif in both binding HSP20 and inhibiting the binding of PDE4D. The importance of this lysine residue is demonstrated by the inability of FQNLTR (SEQ ID NO: 67), which is shown to bind HSP20 in FIG. 8E, to increase HSP20 phosphorylation in SH-SY5Y cells. N-terminal residues may also play a role in disrupting the HSP20-PDE4D complex, as YQNLTK (SEQ ID NO: 75) had no effect on phosphorylation levels, in contrast to FQNLTK (SEQ ID NO: 4). The negative control peptides YQNLTA (SEQ ID NO: 72) and FQNLTA (SEQ ID NO: 49) had similar levels of pS16-Hsp20 expression levels as the controls.

A second group of peptides was also tested for their ability to increase levels of pS16-Hsp20. Two of the candidate disrupters shown to bind to HSP20 in the array (FIG. 8B) induced an average increase when compared with DMSO only controls (GFRLL (SEQ ID NO: 76) and KFKLL (SEQ ID NO: 77)). However, peptides based on the native sequence, FKLLQ (SEQ ID NO: 43), GFKLL (SEQ ID NO: 42), KLL and GFK were indistinguishable from DMSO only controls. The SEM values for each assay were higher in the second group of compounds tested than in the first. The negative control for this group of peptides (GFALL) (SEQ ID NO: 80) also had an average value similar to the DMSO only controls.

### Discussion

Small heat shock proteins are often transiently up-regulated in many tissue types following stress stimuli⁴⁷. Recently, one member of the small heat shock family, namely HSP20, has been shown to have a degree of specificity as a protective mediator in cardiac tissue following injury or insult^{2, 8}. Intriguingly, a key facet of the protective mechanism relayed by HSP20 relates to its phosphorylation on Ser16 by PKA^{2, 17}. Techniques that modulate the amount of phosphorylated HSP20 using genetic engineering¹³ or the trans-membrane delivery of pre-phosphorylated HSP20 analogues¹² have proved effective in triggering protective responses.

Here we show that HSP20 can directly interact and sequester members of the cAMP degrading PDE4 family. Using peptide array analyses we have gained insight into the sites of interaction and developed a peptide that can disrupt HSP20:PDE4 complexes. We show that sequestration of PDE4 by HSP20 is essential for maintenance of the non-phosphorylated state of HSP20 under basal cellular conditions, with chemical ablation of PDE4 activity alone being enough to increase cAMP concentrations in the HSP20 cellular compartment (FIG. 5) to induce its PKA phosphorylation at Ser 16 (FIG. 1).

Members of the PDE4 family play a pivotal role in shaping cAMP gradients responsible for specific activation of localised PKA pools²⁴. Discretely localised PDE4 isoforms can act as “sinks” to locally drain cAMP and protect PKA substrates from inappropriate activation²⁴. Presumably, HSP20-sequestered PDE4 protects this chaperone from inappropriate PKA phosphorylation due to fluctations in basal cAMP levels until cAMP concentrations are increased by actions such as β-adrenergic stimulation. Then rising cAMP levels will eventually saturate tethered PDE4 and allow PKA to be activated and phosphorylate HSP20. As Ser16 phosphorylation of HSP20 provides the “switch” that “turns on” the protective ability of HSP20¹⁷, selective inhibition or displacement of the PDE4 pool associated with HSP20 might be expected to release the ‘brake’ on HSP20 phosphorylation, potentially providing a novel route for the development of cardio-protective agents.

Successful strategies that have been employed for the selective attenuation of PDE4 activity specifically anchored to other signalling proteins include siRNA silencing of PDE4s⁴⁸, PDE4 knockout mice⁴⁹, dominant negative PDE4 constructs³⁰ and disrupter peptides that dissociate PDE4 complexes³². Protein partners of PDE4 usually dock to the enzyme via at least two sites on the phosphodiesterase, and this is true for RACK1-PDE4D5³⁶, βarrestin-PDE4D5³⁶, p75 neurotrophin receptor-PDE4A³⁹, the SUMO E3 ligase PiASy-PDE4D⁵⁰, DISC1-PDE4B³⁸ and ERK MAP kinase-PDE4⁵¹ interactions. Here, however, we note that there is no isoform specificity (FIG. 3) an observation that no doubt reflects the single conserved docking site for HSP20 we identify within the conserved PDE4 catalytic unit (FIG. 4). In trying to gain insight into the functional role of the PDE4-HSP20 complex we utilised sequence encompassing this conserved docking site in the PDE4 catalytic domain to develop a cell-permeable peptide disrupter, anticipating that it might effectively disrupt PDE4:HSP20 complexes (FIG. 6).

Here, the HSP20-PDE4 complex disrupter peptide clearly attenuated the interaction between HSP20 and PDE4, which triggered and increase in the Ser16 phosphorylation status of HSP20 under resting conditions, that is without β₂ adrenergic receptor activation (FIG. 6). Further, we show that shorter fragments of peptide 906 such as FQNLTK (SEQ ID NO: 4), LTK and TK trigger an increase in the Ser16 phosphorylation status of HSP20 in the human neuroblastoma cell line SH-SY5Y.

Chronic stimulation of the β-adrenergic axis has been shown to contribute to the progression of heart failure in animal models and human subjects. Increases in the levels of phospho-HSP20 are instrumental for cardio-protective responses that counteract cellular injury, such as interstitial fibrosis and apoptosis arising from constant β-agonist treatment^{10, 18}. Phospho-HSP20 can also attenuate the isoprenaline mediated hypertrophic response by reducing heart to body weight and regulating cross-section size of cardiac myocytes⁹. Here we demonstrate that ‘peptide 906’, via its induction of HSP20 phosphorylation, can attenuate the hypertrophic response in cultured cardiomyocytes and reduce the increase in myocyte size elicited in response to prolonged activation of β-adrenergic receptors (FIG. 7). Displacement strategies for the specific removal of precisely targeted PDE4 isoforms represents a novel route for the development of therapeutic agents and the potential applications of an agent that promotes the phosphorylation of HSP20 are many. Indeed, phospho-peptide analogues of HSP20 are currently being developed for the reversal of delayed decreases in cerebral perfusion following subarachnoid hemorrhage⁵², for the reduction of TGF induced connective tissue growth factor during fibrosis and scarring⁵³, for the prevention of vasospasm in umbilical artery smooth muscle⁵⁴ and for the inhibition of platelet aggregation¹². Peptide 906, which functions via a previously unrecognised mechanism to promote endogenously phosphorylated HSP20 via disruption of endogenous PDE4-HSP20 complexes may also be efficient in the treatment of these disorders.

In summary, we have identified a novel association between HSP20 and PDE4 enzymes, which gates the protective PKA phosphorylation of HSP20. Disruption of this complex and the resulting induction of phosphorylated HSP20 might provide a novel therapeutic strategy for the treatment of various cellular stress related conditions described herein.

### REFERENCES

1. Salinthone S, Tyagi M, Gerthoffer W T. Small heat shock proteins in smooth muscle. Pharmacol Ther. 2008; 19(1):44-54.
2. Qian J, Ren X, Wang X, Zhang P, Jones W K, Molkentin J D, Fan G C, Kranias E G. Blockade of Hsp20 Phosphorylation Exacerbates Cardiac Ischemia/Reperfusion Injury by Suppressed Autophagy and increased Cell Death. Circulation research. 2009.
3. Sun Y, MacRae T H. The small heat shock proteins and their role in human disease. Febs J. 2005; 272(11):2613-2627.
4. Kappe G, Franck E, Verschuure P, Boelens W C, Leunissen J A, de Jong W W. The human genome encodes 10 alpha-crystallin-related small heat shock proteins: HspB1-10. Cell Stress Chaperones. 2003:8(1):53-61.
5. Wilhelmus M M, Boelens W C, Kox M, Meat-Schieman M L, Veerhuis R, de Waal R M, Verbeek M M. Small heat shock proteins associated with cerebral amyloid angiopathy of hereditary cerebral hemorrhage with amyloidosis (Dutch type) induce interleukin-6 secretion. Neurobioi Aging. 2009; 30(2):229-240.
6. Wilhelmus M M, Boelens W C, Otte-Holler I, Kamps B, Kusters B, Maat-Schieman M L, de Waal R M, Verbeek M M. Small heat shock protein HspB8: its distribution in Alzheimer's disease brains and its inhibition of amyloid-beta protein aggregation and cerebrovascular amyloid-beta toxicity. Acta Neuropathol. 2006; 111(2):139-149.
7. Niwa M, Hara A, Taguchi A, Aoki H. Kozawa O, Mori H. Spatiotemporal expression of Hsp20 and its phosphorylation in hippocampal CA1 pyramidal neurons after transient forebrain ischemia. Neurol Res. 2009; 31(7):721-727.
8. Zhu Y H, Ma T M, Wang X. Gene transfer of heat-shock protein 20 protects against ischemia/reperfusion injury in rat hearts. Acta Pharmacol Sin. 2005; 26(10):1193-1200.
9. Fan G C, Yuan Q, Song G, Wang Y, Chen G, Qian J, Zhou X, Lee Y J. Ashraf M, Kranias E G. Small heat-shock protein Hsp20 attenuates beta-agonist-mediated cardiac remodeling through apoptosis signal-regulating kinase 1. Circulation research. 2006:99(11):1233-1242.
10. Fan G C, Chu G, Mitton B, Song Q, Yuan Q, Kranias E G. Small heat-shock protein Hsp20 phosphorylation inhibits beta-agonist-induced cardiac apoptosis. Circulation research. 2004:94(11):1474-1482.
11. Fan G C, Zhou X, Wang X, Song G, Qian J, Nicolaou P, Chen G, Ran X, Kranias E G. Heat shock protein 20 interacting with phosphorylated Akt reduces doxorubicin-triggered oxidative stress and cardiotoxicity. Circulation research. 2008; 103(11):1270-1279.
12. McLemore E C, Tessier D J, Flynn C R, Furnish E J, Komalavilas P, Thresher J S, Joshi L. Stone W M, Fowl R J, Brophy C M, Transducible recombinant small heat shock-related protein, HSP20, inhibits vasospasm and platelet aggregation. Surgery. 2004; 136(3):573-578.
13. Wang X, Zingarelli B, O'Connor M, Zhang P. Adeyemo A, Kranias E G, Wang Y, Fan G C. Overexpression of Hsp20 prevents endotoxin-induced myocardial dysfunction and apoptosis via inhibition of NF-kappaB activation. J Mol Cell Cardiol. 2009; 47(3):382-390.
14. Dohke T, Wada A, Isono T, Fujii M, Yamamoto T, Tsutamoto T, Horie M. Proteomic analysis reveals significant alternations of cardiac small heat shock protein expression in congestive heart failure. J Card Fail. 2006; 12(1):77-84.
15. Tessier D J, Komalavilas P, Panitch A, Joshi L, Brophy C M. The small heat shock protein (HSP) 20 is dynamically associated with the actin cross-linking protein actinin. J Surg Res. 2003; 11(1):152-157.
16. Pipkin W, Johnson J A, Creazzo T L, Burch J, Komalavilas P, Brophy C. Localization, macromolecular associations, and function of the small heat shock-related protein HSP20 in rat heart. Circulation. 2003; 107(3):469-476.
17. Fan G C, Chu G, Kranias E G. Hsp20 and its cardioprotection. Trends Cardiovasc Med. 2005; 15(4):138-141.
18. Nicolaou P, Knoll R, Haghighi K, Fan G C, Dorn G W, 2nd, Hasenfub 6, Kranias E G. Human mutation in the anti-apoptotic heat shock protein 20 abrogates its cardioprotective effects. The Journal of biological chemistry. 2008; 283(48):33465-33471.
19. Komalavilas P, Penn R B, Flynn C R, Thresher J, Lopes L B, Furnish E J, Guo M, Pallero M A, Murphy-Ullrich J E, Brophy C M. The small heat shock-related protein, HSP20, is a cAMP-dependent protein kinase substrate that is involved in airway smooth muscle relaxation. Am J Physiol Lung Cell Mol Physiol. 2008; 294(1):L69-78.
20. Gilmont R R, Somara S, Bitar K N. VIP induces PKA-mediated rapid and sustained phosphorylation of HSP20. Biochem Biophys Res Commun. 2008; 375(4):552-556.
21. Tyson E K. Macintyre D A, Smith R, Chan E C, Read M. Evidence that a protein kinase A substrate, small heat-shock protein 20, modulates myometrial relaxation in human pregnancy. Endocrinology. 2008; 149(12):6157-6165.
22. Dreiza C M, Brophy C M, Komalavilas P, Furnish E J, Joshi L, Pallero M A, Murphy-Ullrich J E, von Rechenberg M, Ho Y S, Richardson B, Xu N, Zhen Y, Peltier J M, Panitch A. Transducible heat shock protein 20 (HSP20) phosphopeptide alters cytoskeletal dynamics. Faseb J. 2005; 19(2):261-263.
23. Flynn C R, Smoke C C, Furnish E, Komalavilas P, Thresher J, Yi Z, Mandarino L J, Brophy C M. Phosphorylation and activation of a transducible recombinant form of human HSP20 in Escherichia coli. Protein expression and purification. 2007; 52(1):50-58.
24. Baillie G S. Compartmentalized signalling: spatial regulation of cAMP by the action of compartmentalized phosphodiesterases. Febs J. 2009; 276(7):1790-1799.
25. Leroy J, Abi-Gerges A, Nikolaev V O, Richter W, Lechene P, Mazet J L, Conti M, Fischmeister R, Vandecasteele G. Spatiotemporal dynamics of beta-adrenergic cAMP signals and L-type Ca2+ channel regulation in adult rat ventricular myocytes: role of phosphodiesterases. Circulation research. 2008; 102(9):1091-1100.
26. Houslay M D, Schafer P, Zhang K Y. Keynote review: phosphodiesterase-4 as a therapeutic target. Drug Discov Today. 2005; 10(22):1503-1519.
27. Houslay M D, Baillie G S, Maurice D H. cAMP-Specific phosphodiesterase-4 enzymes in the cardiovascular system: a molecular toolbox for generating compartmentalized cAMP signaling. Circulation research. 2007; 100(7):950-966.
28. Bolger G B, McCahill A, Huston E, Cheung Y F, McSorley T, Baillie G S, Houslay M D. The unique amino-terminal region of the PDE4D5 cAMP phosphodiesterase isoform confers preferential interaction with beta-arrestins. J Biol Chem. 2003:278(49):49230-49238.
29. Perry S J, Baillie G S, Kohout T A, McPhee I, Magiera M M, Ang K L, Miller W E, McLean A J, Conti M, Houslay M D, Lefkowitz R J. Targeting of cyclic AMP degradation to beta 2-adrenergic receptors by beta-arrestins. Science. 2002; 298(5594):834-836.
30. Baillie G S, Sood A, McPhee I, Gall I, Perry S J. Lefkowitz R J, Houslay M D. beta-Arrestin-mediated PDE4 cAMP phosphodiesterase recruitment regulates beta-adrenoceptor switching from Gs to Gi. Proc Natl Acad Sci USA. 2003; 100(3):940-945.
31. Lehnart S E, Wehrens X H, Reiken S, Warrier S. Belevych A E, Harvey R D, Richter W, Jin S L, Conti M. Marks A R. Phosphodiesterase 4D deficiency in the ryanodine-receptor complex promotes heart failure and arrhythmias. Cell. 2005; 123(1):25-35.
32. Smith K J, Baillie G S, Hyde E I, Li X, Houslay T M, McCahill A, Dunlop A J, Bolger G B, Klussmann E, Adams D R, Houslay M D. 1H NMR structural and functional characterisation of a cAMP-specific phosphodiesterase-4D5 (PDE4D5) N-terminal region peptide that disrupts PDE4D5 interaction with the signalling scaffold proteins, beta-arrestin and RACK1. Cell Signal 2007; 19(12):2612-2624.
33. MacKenzie S J, Baillie G S, McPhee I, MacKenzie C, Seamons R, McSorley T. Millen J, Beard M B, van Heeke G, Houslay M D. Long PDE4 cAMP specific phosphodiesterases are activated by protein kinase A-mediated phosphorylation of a single serine residue in Upstream Conserved Region 1 (UCR). British journal of pharmacology. 2002; 136(3):421-433.
34. Collins D M, Murdoch H, Dunlop A J, Charych E, Baillie G S, Wang Q: Herberg F W. Brandon N, Prinz A, Houslay M D, NdeI1 alters its conformation by sequestering cAMP-specific phosphodiesterase-4D3 (PDE4D3) in a manner that is dynamically regulated through Protein Kinase A (PKA), Cell Signal. 2008.
35. Carlisle Michel J J, Dodge K L, Wong W, Mayer N C, Langeberg L K, Scott J D PKA-phosphorylation of PDE4D3 facilitates recruitment of the mAKAP signalling complex. The Biochemical journal. 2004; 381(Pt 3):587-592.
36. Bolger G B, Baillie G S, Li X, Lynch M J. Herzyk P, Mohamed A, Mitchell L H, McCahill A, Hundsrucker C, Klussmann E, Adams D R, Houslay M D. Scanning peptide array analyses identify overlapping binding sites for the signalling scaffold proteins, beta-arrestin and RACK1, in cAMP-specific phosphodiesterase PDE4D5. The Biochemical journal. 2006; 398(1):23-36.
37. Richter W, Day P. Agrawal R, Bruss M D, Granier S, Wang Y L, Rasmussen S G, Horner K, Wang P, Lei T, Patterson A J, Kobilka B, Conti M. Signaling from beta1- and beta2-adrenergic receptors is defined by differential interactions with PDE4. Embo J. 2008:27(2):384-393.
38. Millar J K, Pickard B S, Mackie S, James R, Christie S, Buchanan S R, Malloy M P, Chubb J E, Huston E. Baillie G S, Thomson P A, Hill E V. Brandon N J, Rain J C, Camargo L M, Whiting P J, Houslay M D, Blackwood D H, Muir W J, Porteous D J. DISC1 and PDE4B are interacting genetic factors in schizophrenia that regulate cAMP signaling. Science. 2005; 310(5751):1187-1191.
39. Sachs B D, Baillie G S, McCall J R. Passino M A, Schachtrup C, Wallace D A, Dunlop A J, MacKenzie K F, Klussmann E, Lynch M J, Sikorski S L, Nuriel T, Tsigelny I, Zhang J, Houslay M D. Chao M V, Akassoglou K. p75 neurotrophin receptor regulates tissue fibrosis through inhibition of plasminogen activation via a PDE4/cAMP/PKA pathway. J Cell Biol. 2007; 177(6):1119-1132.
40. Terrenoire C, Houslay M D, Baillie G S, Kass R S. The Cardiac IKs Potassium Channel Macromolecular Complex Includes the Phosphodiesterase PDE4D3. The Journal of biological chemistry. 2009; 284(14):9140-9146.
41. Baillie G S, Adams D R, Bhari N, Houslay T M, Vadrevu S, Meng D, Li X, Dunlop A, Milligan G, Bolger G B, Klussmann E, Houslay M D. Mapping binding sites for the PDE4D5 cAMP-specific phosphodiesterase to the N- and C-domains of beta-arrestin using spot-immobilized peptide arrays. The Biochemical journal. 2007; 404(1):71-80.
42. Ponsioen B, Zhao J, Riedl J, Zwartkruis F, van der Krogt G, Zaccolo M, Moolenaar W H, Bos J L, Jalink K. Detecting cAMP-induced Epac activation by fluorescence resonance energy transfer: Epac as a novel cAMP indicator. EMBO reports. 2004; 5(12): 1176-1180,
43. Meng D, Lynch M J, Huston E, Beyermann M, Eichhorst J, Adams D R, Klusmann E, Houslay M D, Baillie G S. MEK1 Binds Directly to {beta}Arrestin1, influencing Both Its Phosphorylation by ERK and the Timing of its isoprenaline-stimulated Internalization. The Journal of biological chemistry. 2009; 284(17):11425-11435.
44. Vistejnova L, Dvorakova J, Hasova M, Muthny T, Velebny V, Soucek K, Kubala L. The comparison of impedance-based method of cell proliferation monitoring with commonly used metabolic-based techniques. Neuro endocrinology letters. 2009; 30(Suppl): 121-127.
45. Urcan E, Haertel U, Styllou M, Hickel R, Scherthan H, Reichl F X. Real-time xCELLigence impedance analysis of the cytotoxicity of dental composite components on human gingival fibroblasts. Dent Mater. 26(1):51-58.
46. Yu N, Atienza J M, Bernard J, Bianc S, Zhu J, Wang X, Xu X, Abassi Y A. Real-time monitoring of morphological changes in living cells by electronic cell sensor arrays: an approach to study G protein-coupled receptors. Analytical chemistry. 2006; 78(1):35-43.
47. Benjamin I J, McMillan D R. Stress (heat shock) proteins: molecular chaperones in cardiovascular biology and disease. Circulation research. 1998; 83(2):117-132.
48. Lynch M J, Baillie G S, Mohamed A, Li X, Maisonneuve C, Klussmann E, van Heeke G, Houslay M D. RNA silencing identifies PDE4D5 as the functionally relevant cAMP phosphodiesterase interacting with beta arrestin to control the protein kinase A/AKAP79-mediated switching of the beta2-adrenergic receptor to activation of ERK in HEK293B2 cells. The Journal of biological chemistry. 2005; 280(39):33178-33189.
49. Bruss M D, Richter W, Horner K, Jin S L, Conti M. Critical role of PDE4D in beta2-adrenoceptor-dependent cAMP signaling in mouse embryonic fibroblasts. The Journal of biological chemistry. 2008:283(33):22430-22442,
50. Li X. Vadrevu S, Dunlop A, Day J. Advent N, Troeger J, Klussmann E, Jaffrey E, Hay R T, Adams D R, Houslay M D, Baillie G S. Selective SUMO modification of cAMP-specific phosphodiesterase-4D5 (PDE4D5) regulates the functional consequences of phosphorylation by PKA and ERK. The Biochemical journal. 428(1):55-65.
51. MacKenzie S J, Baillie G S, McPhee I, Bolger G B, Houslay M D. ERK2 mitogen-activated protein kinase binding, phosphorylation, and regulation of the PDE4D cAMP-specific phosphodiesterases. The involvement of COOH-terminal docking sites and NH2-terminal UCR regions. The Journal of biological chemistry. 2000; 275(22):16609-16617.
52. Furnish E J, Brophy C M, Harris V A, Macomson S, Winger J, Head G A, Shaver E G. Treatment with transducible phosphopeptide analogues of the small heat shock-related protein, HSP20, after experimental subarachnoid hemorrhage: prevention and reversal of delayed decreases in cerebral perfusion. Journal of neurosurgery. 112(3):631-639.
53. Lopes L B, Furnish E J, Komalavilas P, Flynn C R, Ashby P, Hansen A, Ly D P, Yang G P, Longaker M T, Panitch A, Brophy C M. Cell permeant peptide analogues of the small heat shock protein, HSP20, reduce TGF-beta1-induced CTGF expression in keloid fibroblasts. The Journal of investigative dermatology. 2009; 129(3):590-598.
54. Flynn C R, Brophy C M, Furnish E J, Komalavilas P, Tessier D, Thresher J, Joshi L. Transduction of phosphorylated heat shock-related protein 20, HSP20, prevents vasospasm of human umbilical artery smooth muscle. J Appl Physiol. 2005; 98(5):1836-1845.
55. Nikolaev V O, Bunemann M, Hein L, Hannawacker A, Lohse M J. Novel single chain cAMP sensors for receptor-induced signal propagation. The Journal of biological chemistry 2004; 279(36):37215-37218.
56. MacKenzie S J, Baillie G S, McPhee I, MacKenzie C, Seamons R, McSorley T, Millen J, Beard M B, van Heeke G, Houslay M D. Long PDE4 cAMP specific phosphodiesterases are activated by protein kinase A-mediated phosphorylation of a single serine residue in Upstream Conserved Region 1 (UCR1). British journal of pharmacology. 2002; 136(3):421-433.
57. Atienza J M, Yu N, Kirstein S L, Xi B, Wang X, Xu X, Abassi Y A. Dynamic and label-free cell-based assays using the real-time cell electronic sensing system. Assay and drug development technologies. 2006; 4(5):597-607.

1. A method for increasing HSP20 activation in a biological system, comprising contacting the system with an antagonist capable of inhibiting PDE4 binding to HSP20.

2. The method according to claim 1, wherein the antagonist comprises an HSP20-binding moiety capable of inhibiting PDE4 binding to HSP20, wherein the HSP20-binding moiety is a fragment of PDE134 or analogue thereof.

3. The method according to claim 2, wherein the HSP20-binding moiety comprises an amino acid sequence represented by a formula selected from:
X491-X492, X490-X491-X492, X489-X490-X491-X492, X488-X489-X490-X491-X492 or X487-X488-X489-X489-X490-X491-X492, wherein:
X487 is F or is substituted with any amino acid other than Y, optionally an uncharged amino acid or a non-polar amino acid;
X488 is Q or is substituted with any amino acid, optionally an uncharged amino acid or an uncharged polar amino acid;
X489 is N or is substituted with any amino acid, optionally an uncharged amino acid or an uncharged polar amino acid;
X490 is L or is substituted with any amino acid, optionally an uncharged amino acid or a non-polar amino acid;
X491 is T or is substituted with any amino acid, optionally an uncharged amino acid or an uncharged polar amino acid;
X492 is K.

4. The method according to claim 3, wherein:
X487 is F or is substituted with an uncharged amino acid other than Y, or a non-naturally occurring analogue of phenylalanine, optionally a non-polar amino acid;
488 is Q or is substituted with an uncharged amino acid, optionally an uncharged polar amino acid;
X489 is N or is substituted with an uncharged amino acid, optionally an uncharged polar amino acid;
X490 is L or is substituted with an uncharged amino acid, optionally a non-polar amino acid;
X491 is T or is substituted with an uncharged amino acid, optionally an uncharged polar amino acid;
X492 is K.

5. The method according to claim 3, wherein
X487 is F, M, I or L;
X488 is Q or N;
X489 is N or Q;
X490 is L, I or M;
X491 is T or S;
X492 is K.

6. The method according to claim 3, wherein
X487 is F;
X488 is Q;
X489 is N;
X490 is L;
X491 is T;
X492 is K.

7. The method according to claim 2, wherein the HS20-binding moiety comprises an amino acid sequence represented by the formula:
X468-X469-X470-X471-X472-X473-X474-X475-X476-X477-X478-X479-X480-X481-X482-X483-X484-X485-X486-X487-X488-X489-X490-X491-X492, wherein
X468 is E or is substituted with any amino acid, optionally an uncharged amino acid or a negatively charged amino acid;
X469 is N or is substituted with any amino acid, optionally an uncharged amino acid, a negatively charged amino acid, or a negatively charged amino acid;
X470 is H or is substituted with a positively charged amino acid;
X471 is H or is substituted with a positively charged amino acid;
X472 is L or is substituted with any amino acid, optionally an uncharged amino acid or a non-polar amino acid;
X473 is A or is substituted with any amino acid, optionally an uncharged amino acid or a non-polar amino acid;
X474 is V or is substituted with any amino acid, optionally an uncharged amino acid or a non-polar amino acid;
X475 is G or is substituted with any amino acid, optionally an uncharged amino acid or a non-polar amino acid;
X476 is F or is substituted with any amino acid, optionally an uncharged amino acid or a non-polar amino acid;
X477 is K or is substituted with a positively charged amino acid;
X478 is L or is substituted with any amino acid, optionally an uncharged amino acid or a non-polar amino acid;
X479 is L or is substituted with any amino acid, optionally an uncharged amino acid or a non-polar amino acid;
X480 is Q or is substituted with any amino acid, optionally an uncharged amino acid or an uncharged polar amino acid;
X481 is E or is substituted with a negatively charged amino acid or an uncharged amino acid;
X482 is E or is substituted with a negatively charged amino acid or an uncharged amino acid;
X483 is N or is substituted with any amino acid, optionally an uncharged amino acid or an uncharged polar amino acid;
X484 is C or is substituted with any amino acid, optionally an uncharged amino acid or a non-polar amino acid;
X485 is D or is substituted with a negatively charged amino acid;
X486 is I or is substituted with any amino acid, optionally an uncharged amino acid or a non-polar amino acid;
X487 is F or is substituted with any amino acid, optionally an uncharged amino acid or a non-polar amino acid;
X488 is Q or is substituted with any amino acid, optionally an uncharged amino acid or an uncharged polar amino acid;
X489 is N or is substituted with any amino acid, optionally an uncharged amino acid or an uncharged polar amino acid;
X490 is L or is substituted with any amino acid, optionally an uncharged amino acid or a non-polar amino acid;
X491 is T or is substituted with any amino acid, optionally an uncharged amino acid or an uncharged polar amino acid;
X492 is K or is substituted with any amino acid, optionally a positively charged amino acid;
or a fragment thereof which is capable of inhibiting PDE4 binding to HSP20.

8. The method according to claim 7, wherein
X468 is E or is substituted with an uncharged amino acid or a negatively charged amino acid, optionally a non-naturally occurring analogue of glutamate;
X469 is N or is substituted with an uncharged amino acid, optionally an uncharged polar amino acid;
X470 is H or is substituted with a positively charged amino acid;
X471 is H or is substituted with a positively charged amino acid;
X472 is L or is substituted with an uncharged amino acid, optionally a non-polar amino acid;
X473 is A or is substituted with an uncharged amino acid, optionally a non-polar amino acid;
X474 is V or is substituted with an uncharged amino acid, optionally a non-polar amino acid;
X475 is G or is substituted with an uncharged amino acid, optionally a non-polar amino acid, optionally a non-naturally occurring analogue of glycine;
X476 is F or is substituted with an uncharged amino acid or a non-naturally occurring analogue of phenylalanine, optionally a non-polar amino acid;
X477 is K or is substituted with a positively charged amino acid;
X478 is L or is substituted with an uncharged amino acid, optionally a non-polar amino acid;
X479 is L or is substituted with an uncharged amino acid, optionally a non-polar amino acid;
X480 is Q or is substituted with an uncharged amino acid, optionally an uncharged polar amino acid;
X481 is E or is substituted with a negatively charged amino acid or a non-naturally occurring analogue of glutamate;
X482 is E or is substituted with a negatively charged amino acid or a non-naturally occurring analogue of glutamate;
X483 is N or is substituted with an uncharged amino acid, optionally an uncharged polar amino acid;
X484 is C or is substituted with an uncharged amino acid, optionally a non-polar amino acid;
X485 is D or is substituted with a negatively charged amino acid;
X486 is I or is substituted with an uncharged amino acid, optionally a non-polar amino acid;
X487 is F or is substituted with an uncharged amino acid or a non-naturally occurring analogue of phenylalanine, optionally a non-polar amino acid;
X488 is Q or is substituted with an uncharged amino acid, optionally an uncharged polar amino acid;
X489 is N or is substituted with an uncharged amino acid, optionally an uncharged polar amino acid;
X490 is L or is substituted with an uncharged amino acid, optionally a non-polar amino acid;
X491 is T or is substituted with an uncharged amino acid, optionally an uncharged polar amino acid;
X492 is K or is substituted with a positively charged amino acid.

9. The method according to claim 7, wherein
X468 is E or D;
X469 is N or Q;
X470 is H, K or R;
X471 is H, K or R;
X472 is L, I or M;
X473 is A, G or S;
X474 is V, I or A;
X475 is G or A;
X476 is F, M, I or L;
X477 is K or R;
X478 is L, I or M;
X479 is L, I or M;
X480 is Q or N;
X481 is E or D;
X482 is E or D;
X483 is N or Q;
X484 is C, A or G;
X485 is D or E;
X486 is I, V or L;
X487 is F, M, I or L:
X488 is Q or N;
X489 is N or Q;
X490 is L, I or M;
X491 is T or S;
X492 is K or R.

10. The method according to claim 7, wherein the HSP20-binding moiety comprises the amino acid sequence X477-X478-X479-X480-X481.

11. The method according to claim 7, wherein the HSP20-binding moiety comprises at least 6 consecutive amino acids from the amino acid sequence X477-X478-X479-X480-X481-X482-X483-X484-X485-X486-X487-X488-X489-X490-X491-X492.

12. The method according to claim 7, wherein the HSP20-binding moiety comprises at least 10 consecutive amino acids from the amino acid sequence X469-X470-X471-X472-X473-X474-X475-X476-X477-X478-X479-X480-X481-X482-X483-X484-X485-X486-X487-X488-X489-X490-X491-X492.

13. The method according to claim 7, wherein the amino acid sequence X477-X478-X479-X480-X481 is KLLQE (SEQ ID NO: 5).

14. The method according to claim 7, wherein the HSP20-binding moiety comprises the amino acid sequence KLLQEENCDIFQNLTK (SEQ ID NO: 16).

15. The method according to claim 7, wherein the HSP20-binding moiety comprises the amino acid sequence GFRLL (SEQ ID NO: 76) or KFKLL (SEQ ID NO: 77).

16. The method according to claim 3, wherein the HSP20-binding moiety comprises and N-terminal acetyl group and/or a C-terminal acetyl group.

17. The method according to claim 2, wherein the HSP20-binding moiety comprises the catalytic domain of PDE4.

18. The method according to claim 2, wherein the HSP20-binding moiety has of a maximum of 5, 10, 20, or 50 amino acids.

19. The method according to claim 1, wherein the antagonist comprises an N-terminal amino acid capping group formed by a condensation reaction with any one of the following:

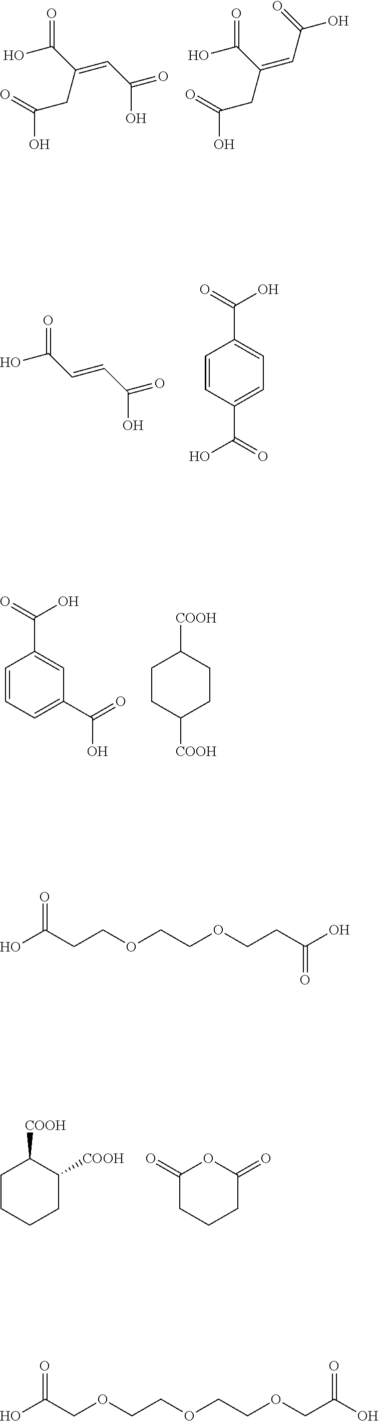

20. The method according to claim 1, wherein the antagonist comprises a heterologous component.

21. The method according to claim 20, wherein the heterologous component comprises a moiety to modulate at least one of the stability, activity, immunogenicity, solubility, bioavailability, membrane permeability or localisation of the antagonist.

22. The method according to claim 20, wherein the heterologous component comprises a peptoid moiety.

23. A method according to claim 1 performed in vitro.

24. A method according to claim 23, wherein the biological system comprises an isolated tissue, blood, plasma or serum sample, or an isolated cell.

25. A method of treating a pathological condition in an individual, comprising administering an antagonist capable of inhibiting PDE4 binding to HSP20 to said individual.

26. (canceled)

27. (canceled)

28. The method of claim 25, wherein the antagonist comprises the HSP20-binding moiety described in claim 2.

29. The method of claim 25, wherein the pathological condition is selected from cerebral amyloid angiopathy, Alzheimer's disease, forebrain ischemia, cardiac disease, cardiac ischemia/reperfusion, chronic beta-adrenergic stimulation, pharmacological treatment by doxorubicin, vasospasm, platelet aggregation, endotoxin induced myocardial dysfunction, congestive heart failure, ischemia/reperfusion injury, apoptosis, cell necrosis, scar tissue formation, fibrotic disorders, interstitial fibrosis and apoptosis arising from constant β-agonist treatment, and delayed decreases in cerebral perfusion following subarachnoid hemorrhage.

30. The method of claim 25, wherein the treatment is prophylactic.

31. A method of testing a candidate agent for an ability to inhibit binding between PDE4 and HSP20, comprising contacting HSP20 or a fragment thereof with
(i) PDE4 or a fragment thereof; and
(ii) the candidate agent;
and determining the binding of PDE4 or a fragment thereof with HSP20 or a fragment thereof between (i) and (ii).

32. The method according to claim 31, wherein the HSP20 or fragment thereof, or PDE4 or a fragment thereof is immobilised on a solid phase.

33. A method of screening for an agent capable of inhibiting binding between PDE4 and HSP20, the method comprising
(i) providing a candidate agent; and
(ii) testing the candidate agent for the ability to inhibit binding between PDE4 or a fragment thereof and HSP20 or a fragment thereof.

34. The method according to of claim 31, wherein the candidate agent is a fragment of HSP20.

35. A method of screening for an agent capable of inhibiting binding between PDE4 and HSP20, the method comprising
(i) providing a candidate agent; and
(ii) testing the candidate agent for the ability to inhibit binding between an HSP20-binding moiety and HSP20 or a fragment thereof.

36. A method of testing a candidate agent for an ability to inhibit binding between PDE4 and HSP20, comprising contacting the candidate agent with
(i) HSP20 or a fragment thereof; and
(ii) an HSP20-binding moiety capable of binding HSP20;
and determining binding between (i) and (ii).

37. The method according to claim 35, wherein the HSP20-binding moiety is the HSP20-binding moiety described in claim 2.

38. The method according to claim 35, wherein the HSP20-binding moiety or HSP20 or fragment thereof is immobilised on a solid phase.

39. The method of claim 31, wherein the candidate agent is a small molecule or peptidomimetic.

40. A method of optimising an HSP20-binding moiety for the ability to inhibit binding between PDE4 and HSP20, comprising
(i) providing a parent HSP20-binding moiety capable of inhibiting binding between PDE4 and HSP20,
(ii) making a variant moiety of the parent HSP20-binding moiety by substituting, deleting or inserting one or more amino acids,
(iii) testing the variant moiety for the ability to inhibit binding between PDE4 and HSP20, and
(iv) comparing the ability of the variant moiety to inhibit binding between PDE4 and HSP20 to the ability of the parent HSP20-binding moiety.

41. The method according to claim 40, wherein the parent HSP20-binding moiety is the HSP20-binding moiety described in claim 2.

42. An antagonist capable of inhibiting binding between PDE4 and HSP20, as described in claim 3, with the proviso that the antagonist does not consist of amino acid residues 461 to 485, 466 to 490, 471 to 495 or 476 to 500 of PDE4D5, or a full length PDE4 protein.

43. The antagonist according to claim 42, consisting of the HSP20-binding moiety described in claim 3.

44. An isolated nucleic acid encoding the antagonist described by or according to claim 42.

45. A vector comprising a nucleic acid according to claim 44.

46. A host cell comprising a nucleic acid according to claim 44.

47. (canceled)

48. A pharmaceutical composition comprising an antagonist according to claim 1 and a pharmaceutically acceptable carrier.

49. An isolated nucleic acid encoding the antagonist described by or according to claim 1.

50. A vector comprising a nucleic acid according to claim 49.

51. A host cell comprising a nucleic acid according to claim 49.

52. A pharmaceutical composition comprising a nucleic acid according to claim 49.

53. A pharmaceutical composition comprising a nucleic acid according to claim 44.
